(19) United States
(12) Patent Application Publication
(10) Pub. No.: US 2008/0039639 A1
(43) Pub. Date: Feb. 14, 2008

(54) Chromane and chromene derivatives and uses thereof

(75) Inventors: Alexander V. Gontcharov, Rivervale, NJ (US); Antonia A. Nikitenko, Tarrytown, NY (US); Panolil Raveendranath, Monroe, NY (US); Chia-Cheng Shaw, Ville Saint-Laurent (CA); Bogdan K. Wilk, New City, NY (US); Dahui Zhou, East Brunswick, NJ (US)

Correspondence Address: CHOATE, HALL & STEWART LLP/WYETH, PATENT GROUP, TWO INTERNATIONAL PLACE, BOSTON, MA, 02110, US

(73) Assignee: Wyeth, Madison, NJ (US)

(21) Appl. No.: 11/787,860

(22) Filed: Apr. 18, 2007

Related U.S. Application Data
(60) Provisional application No. 60/792,913, filed on Apr. 18, 2006.
(60) Provisional application No. 60/854,507, filed on Oct. 25, 2006.

(51) Int. Cl. C07D 311/04

(52) U.S. Cl. 549/402; 549/406; 549/407

(57) ABSTRACT

Methods of preparing compounds of formula I or pharmaceutically acceptable salts thereof are provided: 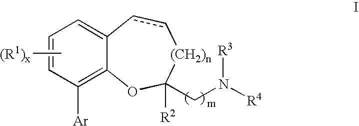
wherein each of R¹, R², R³, R⁴, x, m, n, and Ar are as defined, and described in classes and subclasses herein, which are agonists or partial agonists of the 2C subtype of brain serotonin receptors. The compounds, and compositions containing the compounds, can be used to treat a variety of central nervous system disorders such as schizophrenia.

### CROSS-REFERENCE TO RELATED APPLICATIONS

The present invention claims prior to U.S. provisional patent application Ser. No. 60/792,913, filed Apr. 18, 2006, and U.S. provisional patent application Ser. No. 60/854,507, filed Oct. 25, 2006, the entirety of each of which is hereby incorporated herein by reference.

### FIELD OF THE INVENTION

The present invention relates to 5-HT_{2C} receptor agonists or partial agonists, processes for their preparation, and uses thereof.

### BACKGROUND OF THE INVENTION

Schizophrenia affects approximately 5 million people. The most prevalent treatments for schizophrenia are currently the ‘atypical’ antipsychotics, which combine dopamine (D₂) and serotonin (5-HT_{2A}) receptor antagonism. Despite the reported improvements in efficacy and side-effect liability of atypical antipsychotics relative to typical antipsychotics, these compounds do not appear to adequately treat all the symptoms of schizophrenia and are accompanied by problematic side effects, such as weight gain (Allison, D. B., et. al., Am. J. Psychiatry, 156: 1686-1696, 1999; Masand, P. S., Exp. Opin. Pharmacother. I: 377-389, 2000; Whitaker, R., Spectrum Life Sciences. Decision Resources. 2:1-9, 2000).

Atypical antipsychotics also bind with high affinity to 5-HT_{2C} receptors and function as 5-HT_{2C} receptor antagonists or inverse agonists. Weight gain is a problematic side effect associated with atypical antipsychotics such as clozapine and olanzapine, and it has been suggested that 5-HT_{2C} antagonism is responsible for the increased weight gain. Conversely, stimulation of the 5-HT_{2C} receptor is known to result in decreased food intake and body weight (Walsh et. al., Psychopharmacology 124: 57-73, 1996; Cowen, P. J., et. al., Human Psychopharmacology 10: 385-391, 1995; Rosenzweig-Lipson, S., et. al., ASPET abstract, 2000).

Several lines of evidence support a role for 5-HT_{2C} receptor agonism or partial agonism as a treatment for schizophrenia. Studies suggest that 5-HT_{2C} antagonists increase synaptic levels of dopamine and may be effective in animal models of Parkinson's disease (Di Matteo, V., et. al., Neuropharmacology 37: 265-272, 1998; Fox, S. H., et. al., Experimental Neurology 151: 35-49, 1998). Since the positive symptoms of schizophrenia are associated with increased levels of dopamine, compounds with actions opposite to those of 5-HT_{2C} antagonists, such as 5-HT_{2C} agonists and partial agonists, should reduce levels of synaptic dopamine. Recent studies have demonstrated that 5-HT_{2C} agonists decrease levels of dopamine in the prefrontal cortex and nucleus accumbens (Millan, M. J., et. al., Neuropharmacology 37: 953-955, 1998; Di Matteo, V., et. al., Neuropharmacology 38: 1195-1205, 1999; Di Giovanni, G., et. al., Synapse 35: 53-61, 2000), brain regions that are thought to mediate critical antipsychotic effects of drugs like clozapine. However, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in the substantia nigra. The differential effects of 5-HT_{2C} agonists in the mesolimbic pathway relative to the nigrostriatal pathway suggest that 5-HT_{2C} agonists have limbic selectivity, and will be less likely to produce extrapyramidal side effects associated with typical antipsychotics.

### SUMMARY OF THE INVENTION

As described herein, the present invention provides methods for preparing compounds having activity as 5HT_{2C} agonists or partial agonists. These compounds are useful for treating schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, intellectual deficit associated with Alzheimer's disease, bipolar disorders, depressive disorders, mood episodes, anxiety disorders, adjustment disorders, eating disorders, epilepsy, sleep disorders, migraines, sexual dysfunction, gastrointestinal disorders, obesity and its comorbidities, or a central nervous system deficiency associated with trauma, stroke, or spinal cord injury. Such compounds include those of formula I:

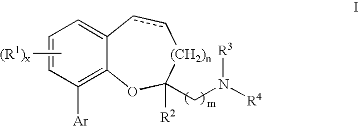

or a pharmaceutically acceptable salt thereof, wherein:
m is 1 or 2;
n is 0 or 1;
[symbol] designates a single or double bond;
Ar is thienyl, furyl, pyridyl, or phenyl, wherein Ar is optionally substituted with one or more R* groups;
each R* is independently -Ph, halogen, —CN, —R or —OR;
each R is independently hydrogen, C₁₋₆ aliphatic or C₁₋₆ haloaliphatic;
x is 0-3;
each R¹ is independently —R, —CN, halogen or —OR;
R² is hydrogen, C₁₋₃ alkyl, or —O(C₁₋₃ alkyl); and
each of R³ and R⁴ is independently hydrogen, C₁₋₆ aliphatic or C₁₋₆ fluoroaliphatic;

The present invention also provides synthetic intermediates useful for preparing such compounds.

### DETAILED DESCRIPTION OF CERTAIN EMBODIMENTS

The methods and intermediates of the present invention are useful for preparing compounds as described in U.S. provisional patent application Ser. No. 60/673,820, filed Apr. 22, 2005, the entirety of which is hereby incorporated herein by reference.

In certain embodiments, the present compounds are generally prepared according to Scheme I set forth below:

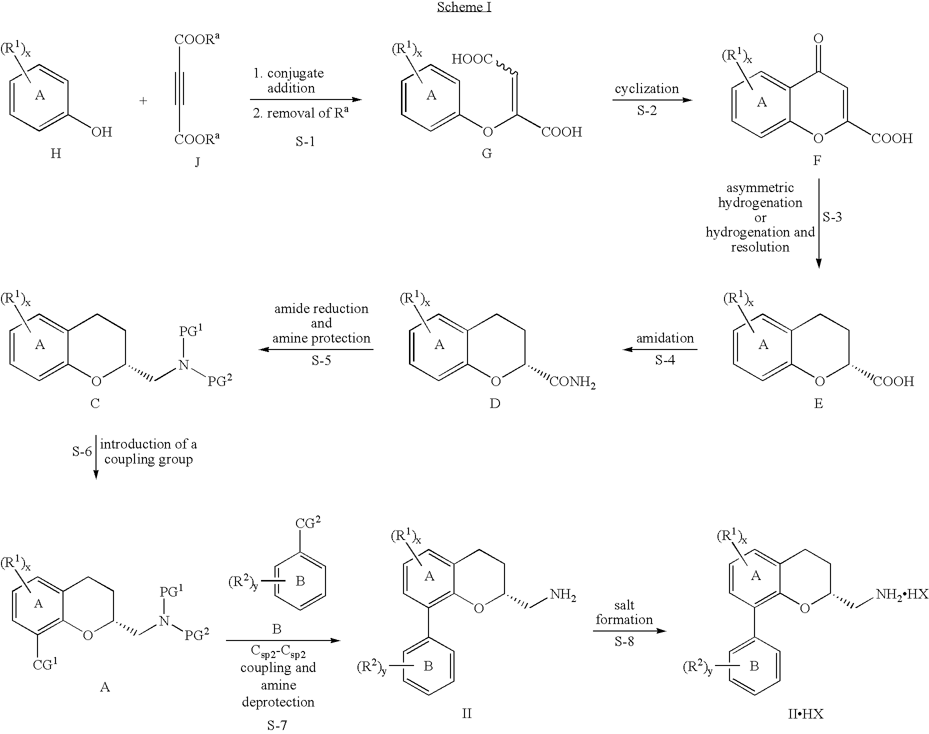

In Scheme I above, each of R¹, R², R^{a}, x, y, PG¹, PG², CG¹, and CG² is as defined below and in classes and subclasses as described herein.

In one aspect, the present invention provides methods for preparing chiral 2,8-disubstituted chromane compounds of formulae A, II, and II•HX in enantiomerically enriched form according to the steps depicted in Scheme I, above.

In step S1 a compound of formula H is allowed to react via conjugate addition with a compound of formula J, following which the R^{a} groups are removed to afford the product of formula G, as depicted in Scheme II, below. One of ordinary skill in the art will appreciate that a wide variety of reaction conditions may be employed to promote this transformation, therefore a wide variety of reaction conditions are envisioned; see generally, March's Advanced Organic Chemistry: Reactions, Mechanisms, and Structure, M. B. Smith and J. March, 5^{th} Edition, John Wiley & Sons, 2001 and Comprehensive Organic Transformaions, R. C. Larock, 2^{nd} Edition, John Wiley & Sons, 1999. For example, the conjugate addition step may be run in the presence or absence of a base, and with or without heating. In certain embodiments, the conjugate addition is run in the presence of potassium carbonate, potassium hydroxide, sodium hydroxide, tetrabutylammonium hydroxide, benzyltrimethylammonium hydroxide, triethylbenzylammonium hydroxide, 1,1,3,3-tetramethylguanidine, 1,8-diazabicyclo[5.4.0]undec-7-ene, N-methylmorpholine, diisopropylethylamine, tetramethylethylenediamine, pyridine, or triethylamine.

In certain embodiments, the reaction is carried out in a suitable medium. A suitable medium is a solvent or a solvent mixture that, in combination with the combined reacting partners and reagents, facilitates the progress of the reaction therebetween. The suitable solvent may solubilize one or more of the reaction components, or, alternatively, the suitable solvent may facilitate the suspension of one or more of the reaction components; see, generally, March (2001). In certain embodiments the present transformation is run in diphenyl ether, dioxane, anisole, acetone, tetrahydrofuran, ethyl acetate, isopropyl acetate, dimethylformamide, ethylene glycol, toluene, water, diisopropylethylamine, triethylamine, pyridine, N-methylmorpholine, acetonitrile, N-methylpyrrolidine, or mixtures thereof. In certain embodiments, the conjugate addition is performed in a mixture of pyridine and dioxane. In other embodiments, no additional solvent is added. In still other embodiments, excess of the phenol (corresponding to formula H) is employed to serve as a solvent. In other embodiments the reaction is conducted at temperatures between about 25° C. and about 110° C. In yet other embodiments, the reaction is conducted at about 25° C. In other embodiments, the conjugate addition is carried out in a manner substantially similar to the procedures outlined in Ruhemann, S. J. Chem. Soc. 1900, 77, 1121, Gudi, M. N. et al. Indian J. Chem. 1969, 7, 971, Cairns, H. et al. J. Med. Chem. 1972, 15, 583, Stoermer, M. J. and Fairlie, D. P. Aust. J. Chem. 1995, 48, 677, and Fitzmaurice, C. et al. British Patent No. 1262078, (filed 24 May, 1968).

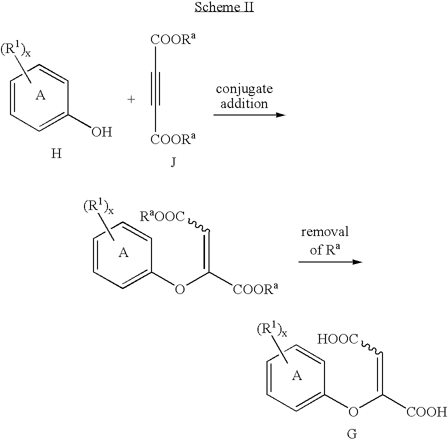

Each R¹ group of formulae H, G, F, E, D, C, A, II, and II•HX is independently —R, —CN, halogen or —OR, wherein each R is independently hydrogen, C₁₋₆ aliphatic or C₁₋₆ haloaliphatic. Examples of suitable R¹ groups include hydrogen, methyl, ethyl, isopropyl, chloro, and fluoro. According to one aspect of the present invention, R¹ is fluoro. According to another aspect of the present invention, R¹ in ring A of compounds of formulae H, G, F, E, D, C, A, II, and II•HX is located at the ring position that corresponds to the position para to OH in formula H.

The numeral x of formulae H, G, F, E, D, C, A, II, and II•HX is 0-3. According to one aspect of the present invention, x is 1.

Each R^{a} group of formula J and of the intermediate compound shown in Scheme 2 is independently hydrogen, C₁₋₆ aliphatic, phenyl, benzyl, or tri(C₁₋₆ aliphatic)silyl. In certain embodiments, each R^{a} is independently selected from ethyl, methyl, hydrogen, tert-butyl, or trimethylsilyl. In other embodiments, each R^{a} is ethyl. One of ordinary skill in the art will recognize that there are a wide variety of reaction conditions that can be employed to remove the R^{a} groups to afford compounds of formula G, therefore, a wide variety of conditions are envisioned; see generally, March, (2001) and Larock (1999). For example, the removal the R^{a} groups can be promoted by reaction with base (e.g., sodium hydroxide, tetrabutylammonium hydroxide, or the like) or acid (e.g., hydrochloric acid, sulfuric acid, acetic acid, camphorsulfonic acid, p-toluenesulfonic acid, or the like), with sources of fluoride (e.g., tetrabutylammonium fluoride, potassium fluoride, pyridinium fluoride, triethylammonium fluoride, tetrabutylammnonium triphenyldifluorosilicate, or the like), and optionally with heating of the reaction mixture. In certain embodiments, the removal of the R^{a} groups is promoted by reaction with sodium hydroxide. In other embodiments, this reaction is conducted at a temperature of between about 40° C. and about 100° C.

At step S2, a compound of formula G is cyclized to afford a compound of formula F. One of ordinary skill in the art will recognize that there are a wide variety of reaction conditions that can be employed to cyclize compounds of formula G, therefore, a wide variety of conditions are envisioned; see generally, March, (2001) and Larock (1999). In certain embodiments, the cyclization is promoted by treating a compound of formula G with a suitable Brønsted acid. Exemplary acids include hydrochloric, sulfuric, phosphoric, polyphosphoric, methanesulfonic, Eaton's reagent (P₂O₅/MeSO₃H), chlorosulfonic, camphorsulfonic, and p-toluenesulfonic. In other embodiments, additional reagents are employed, including, for example, phosphorus pentoxide, phosphorus trichloride, phosphorus pentachloride, acetyl chloride, or acetic anhydride. One of ordinary skill in the art will recognize that some of the conditions described will promote formation of an intermediate acylchloride prior to undergoing cyclization. In yet another embodiment, the reaction is conducted with acetyl chloride or water as solvent. In still other embodiments, the cyclization is conducted as described in Ruhemann (1900), Gudi (1969), Cairns (1972), Stoermer (1995), or Fitzmaurice, C. et al. British Patent No. 1262078, (filed 24 May, 1968).

In step S3, a compound of formula F is reduced to afford a compound of formula E. One of ordinary skill in the art will recognize that compounds of formulae E, D, C, A, II, and II•HX contain a stereogenic carbon. Accordingly, this invention encompasses each individual enantiomer of compounds of formulae E, D, C, A, II, and II•HX as well as mixtures thereof. While a single stereochemical isomer is depicted for formulae E, D, C, A, II, and II•HX in Scheme I, it will be appreciated that mixtures of enantiomers of these formulae are accessible enriched in either enantiomer via the present invention. As used herein, the terms “enantiomerically enriched” and “enantioenriched” denote that one enantiomer makes up at least 75% of the preparation. In certain embodiments, the terms denote that one enantiomer makes up at least 80% of the preparation. In other embodiments, the terms denote that at least 90% of the preparation is one of the enantiomers. In other embodiments, the terms denote that at least 95% of the preparation is one of the enantiomers. In still other embodiments, the terms denote that at least 97.5% of the preparation is one of the enantiomers. In yet another embodiment, the terms denote that the preparation consists of a single enantiomer to the limits of detection (also referred to as “enantiopure”). As used herein, when “enantioenriched” or “enantiomerically enriched” are used to describe a singular noun (e.g., “an enantioenriched compound of formula II” or “an enantioenriched chiral amine”), it should be understood that the “compound” or “acid” may be enantiopure, or may in fact be an enantioenriched mixture of enantiomers. Similarly, when “racemic” is used to describe a singular noun (e.g., “a racemic compound of formula E”), it should be understood that the term is in fact describing a 1:1 mixture of enantiomers.

In one aspect of the present invention, step S3 is carried out by (a) first subjecting the compound of formula F to hydrogenation conditions, (b) forming diastereomeric salts by combining the racemic mixture of the hydrogenation product with an enantioenriched chiral amine, (c) selectively crystallizing one of the diastereomeric salts to afford a diastereomerically enriched mixture of salts, and (d) recovering the acid in enantioenriched form from the diastereomerically enriched salt, as depicted in Scheme III, below. In certain embodiments, the hydrogenation in (a) is conducted in the presence of a palladium catalyst. In other embodiments, the palladium catalyst is palladium on carbon. In still other embodiments, the hydrogenation is run in methanol, ethanol, or acetic acid. According to one aspect of the present invention, the hydrogenation is run in methanol. In yet other embodiments, the hydrogenation is conducted in the presence of sulfuric acid, acetic acid, or both. In some embodiments, the hydrogenation is conducted in the presence of sulfuric acid. In still other embodiments, the hydrogenation is conducted as described in Witiak, D. T. et al. J. Med. Chem. 1975, 18, 934.

In another aspect of the present invention, the enantioenriched chiral amine is (R)-1-phenyl-propylamine, (−)-cinchonidine, or -(−)-1-(1-naphthyl)-ethylamine. In certain embodiments, the enantioenriched chiral amine is (R)-1-phenyl-propylamine.

In certain embodiments, the crystallization in step (c) is conducted in acetonitrile, methanol, ethanol, isopropanol, ethyl acetate, isopropyl acetate, diethyl ether, tert-butyl methyl ether, benzene, toluene, dichloromethane or the like. In certain embodiments, the free acid is liberated in step (d) by treating the salt with hydrochloric acid or sulfuric acid. In other embodiments, step (d) is conducted in toluene, water, or mixtures thereof. In other embodiments, the resolution step is conducted as described in Wigerinck, P. T. B. P. et al., International patent application number WO 9929687 A1 (1999); Van Lommen, G. R. E. et al., European patent application publication number EP 145067 A2 (1985); or Schaff, T. K. et al. J. Med. Chem. 1983, 26, 328.

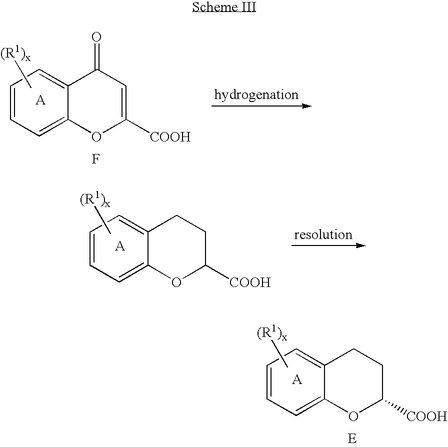

In another aspect of the present invention, step S-3 is carried out by (a) first subjecting the compound of formula F to hydrogenation conditions, (b) resolving the racemic reduced product by enzymatic means. In certain embodiments, the enzymatic resolution is carried out according to Schutt, H., German patent application publication number DE 4430089 A1 (1996); Urban, F. J., European patent application publication number EP 0448254 A2 (1991); and Rossi, R. F., Jr., international patent application publication number WO 9640975 A1 (1996).

In yet another aspect of the present invention, step S-3 is carried out by (a) hydrogenating a compound of formula F in an asymmetric fashion to afford an intermediate ketone-containing compound in enantiomerically enriched form, and (b) hydrogenating said intermediate to reduce the keto moiety and afford a compound of formula E in enantiomerically enriched form, as shown in Scheme IV, below. In certain embodiments, the asymmetric hydrogenation in step (a) is catalyzed by a suitable chiral catalyst. In certain embodiments, the chiral catalyst is a complex comprising a transition metal species and a suitable chiral ligand. In certain embodiments, the transition metal species is a late transition metal species (e.g., a Ru, Rh, Pd, Ir, or Pt species). In other embodiments the transition metal species is a rhodium or ruthenium species. In certain embodiments, the chiral ligand contains a phosphorus moiety that is capable of binding a transition metal species (e.g., a phosphine or phosphite moiety). In other embodiments the chiral ligand contains an olefinic moiety that is capable of binding a transition metal species. In yet other embodiments, the chiral ligand contains a carbene moiety that is capable of binding to a transition metal species. Suitable chiral ligands for asymmetric hydrogenation are well known in the art; see, e.g., Stereochemistry of Organic Compounds, E. L. Eliel and S. H. Silen, 1994, John Wiley and Sons; Asymmetric Catalysis in Organic Synthesis, R. Noyori, 1994, John Wiley and Sons; X. Cui and K. Burgess, Chem. Rev. 2005, 105, 3272; and W. Tang and X. Zhang, Chem. Rev. 2003, 103, 3029. Additional exemplary chiral ligands include, but are not limited to, JosiPhos-type, MandyPhos™-type, WalPhos-type, TaniaPhos™-type, RoPhos-type, DIPAMP-type, Butiphane-type, BPE-type, QUINAP-type, BINAP-type, NorPhos-type, MonoPhos™-type, TunePhos-type, MalPhos-type, DuPhos-type, PHOX-type, KetalPhos-type, f-KetalPhos-type, TangPhos-type, BIPHEP-type, ferrotane-type, Binaphane-type, f-Binaphane-type, Binapine-type, FAP-type, MOP-type, DIOP-type, ChiraPhos-type, BPPM-type, and BICP-type. The term “asymmetric hydrogenation,” as used herein refers to the hydrogenation of an achiral or chiral substrate which results in an enantiomerically enriched chiral product. In certain embodiments the asymmetric hydrogenation is catalyzed by a chiral transition metal-containing species. In certain embodiments, the hydrogenation in step (b) is is conducted in the presence of a palladium catalyst. In other embodiments, the palladium catalyst is palladium on carbon. In still other embodiments, the hydrogenation is run in methanol. In yet other embodiments, the hydrogenation is conducted in the presence of sulfuric acid and acetic acid.

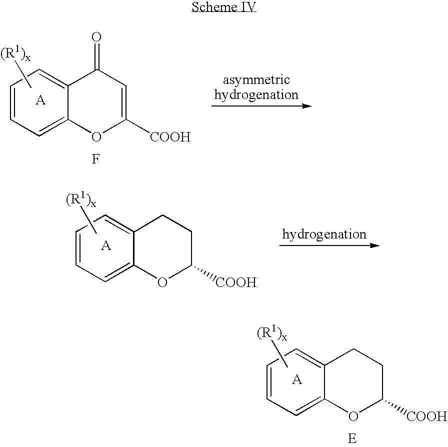

In certain embodiments, the chiral ligand employed in the asymmetric hydrogenation is selected from those depicted in Table I. In other embodiments, the chiral ligand is WalPhos W008-1.

TABLE I
Representative catalysts

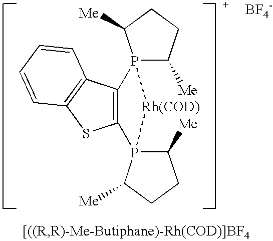

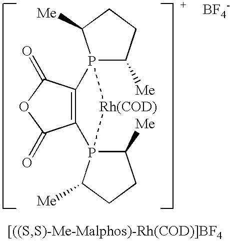

Representative ligands

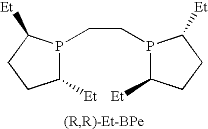

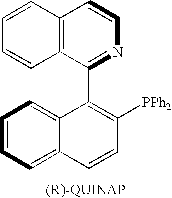

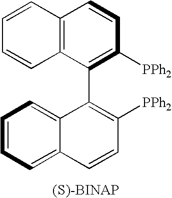

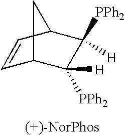

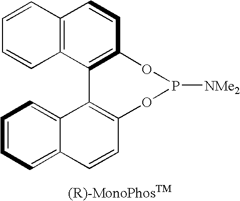

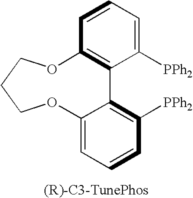

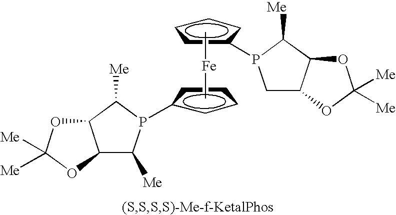

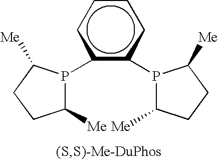

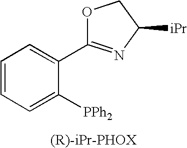

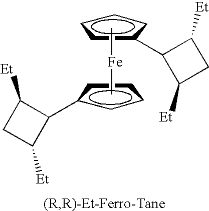

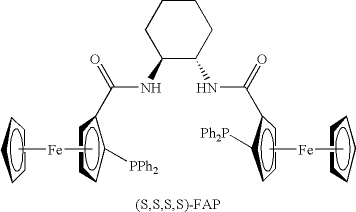

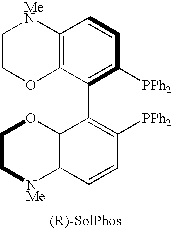

JosiPhos-type

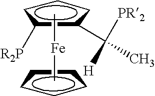

J001-1: R = Ph, R′ = cyclohexyl
J002-1: R = Ph, R′ = t-Bu
J002-2: R = Ph, R′ = t-Bu [stereochemistry opposite to that depicted]
J003-1: R = cyclohexyl, R′ = cyclohexyl
J005-1: R = Ph, R′ = 3,5-dimethyiphenyl
J006-1: R = 3,5-di(trifluoromethyl)phenyl, R′ = cyclohexyl
J007-1: R = 3,5-dimethyl-4-methoxyphenyl, R′ = cyclohexyl
J008-1: R = 3,5-di(trifluoromethyl)phenyl, R′ = 3,5-dimethylphenyl
J009-1: R = cyclohexyl, R′ = t-Bu
J011-1: R = 4-trifluoromethylphenyl, R′ = t-Bu
J012-1: R = para-tolyl, R′ = t-Bu
J013-1: R = 3,5-dimethyl-4-methoxyphenyl, R′ = t-Bu
J015-2: R = 2-furyl, R′ = cyclohexyl
[stereochemistry opposite to that depicted]
J031-1: R = phenyl, R′ = cyclopentyl
J202-2: R = 4-methoxyphenyl, R′ = t-Bu
[stereochemistry opposite to that depicted]
J211-1: R = 2-methylphenyl, R′ = t-Bu
J212-2: R = 2-furyl, R′ = t-Bu [stereochemistry opposite to that depicted]
J216-1: R = 1-naphthyl, R′ = t-Bu
J216-2: R = 1-naphthyl,
R′ = t-Bu [stereochemistry opposite to that depicted]

WalPhos-type

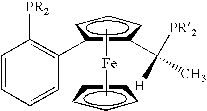

W003-1: R = Ph, R′ = cyclohexyl
W006-1: R = Ph, R′ = 3,5-dimethylphenyl
W008-1: R = cyclohexyl, R′ = 3,5-di(trifluoromethyl)phenyl
W008-2: R = cyclohexyl, R′ = 3,5-di(trifluoromethyl)phenyl
[stereochemistry opposite to that depicted]

TaniaPhosTM-type

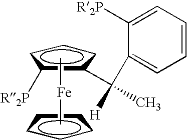

T001-1: R = dimethyamino, R′ = Ph, R″ = Ph
T002-1: R = dimethyamino, R′ = cyclohexyl, R″ = cyclohexyl

MandyPhosTM-type

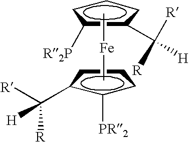

M001-1: R = dimethyamino, R′ = Ph, R″ = Ph
M002-1: R = dimethyamino, R′ = Ph, R″ = cyclohexyl
M004-1: R = dimethyamino, R′ = Ph,
R″ = 3,5-dimethyl-4-methoxyphenyl

According to another aspect of the present invention, the hydrogenation reactions in step S-3, described above and herein, are conducted at pressures at about 50 psi or above. In certain embodiments, the hydrogenations are conducted with heating of the reaction mixture. In other embodiments, the hydrogenations are conducted at temperatures between about 30° C. and about 50° C.

In step S-4, a compound of formula E is amidated to afford a compound of formula D. One of ordinary skill in the art will recognize that there are a wide variety of reaction conditions that can be employed to amidate compounds of formula G, therefore, a wide variety of conditions are envisioned; see generally, March (2001); Larock (1999); Benz, G. “Synthesis of Amides and Related Compounds.” in Comprehensive Organic Synthesis, Trost, B. M., Editor, Pergamon Press: New York, N.Y., Vol. 6; and Bailey, P. D. et al. “Amides” in Comprehensive Organic Functional Group Transformation, Katritzky, et. al. Editors, Pergamon: New York, N.Y., Vol. 5. In certain embodiments, the amidation is conducted by first activating the carboxylic acid to facilitate acylation (e.g., by reaction with SOCl₂ or similar reagents), and subsequently treating the activated species with a source of ammonia [e.g., ammonia gas or solution in tetrahydrofuran toluene, heptane, tert-butyl methyl ether, diethyl ether, ethyl acetate, isopropyl acetate, dichloromethane, chloroform, dichloroethane, or water (e.g., NH₄OH)]. In other embodiments, this reaction is conducted by first activating the carboxylic acid to facilitate acylation by reaction with SOCl₂ and subsequently treating the activated species with NH₄OH. In still other embodiments, the reaction is run in toluene, benzene, ethyl acetate, dichloromethane, chloroform, dichloroethane, combinations thereof. In some embodiments, the reaction is run in the absence of solvent. In other embodiments, the reaction is run at a temperature between about −10° C. and 150° C. In still other embodiments, the reaction is run at a temperature between about 50° C. and about 100° C. In yet other embodiments, the reaction is conducted in a manner substantially similar to that described in Zhang, M. et al. Tetrahedron Lett. 2004, 45, 5229 or Devant, R. International patent application publication number WO05037817 (2005).

In step S-5, the amide moiety in compounds of formula D is reduced to an amine, and the resulting amine is optimally protected to afford compounds of formula C. In compounds of formulae C and A, PG¹ and PG² are each independently hydrogen or amino protecting groups. Protected amines are well known in the art and include those described in detail in Greene (1999). Suitable mono-protected amines have to withstand the reaction conditions of the next step unchanged and may further include, but are not limited to, aralkylamines, carbamates, allyl amines, and the like. Examples of suitable mono-protected amino moieties include t-butyloxycarbonylamino (—NHBOC), ethyloxycarbonylamino, methyloxycarbonylamino, allyloxycarbonylamino (—NHAlloc), benzyloxocarbonylamino (—NHCBZ), allylamino, benzylamino (—NHBn), formamido, and the like. Suitable di-protected amines include amines that are substituted with two substituents independently selected from those described above as mono-protected amines, and further include Suitable di-protected amines also include pyrroles and the like. In certain embodiments, one of either PG¹ or PG² or both in compounds of formulae C and A may be hydrogen. The amino group can also be masked as an azido group —N₃. According to one aspect of the invention, the —N(PG¹)(PG²) moiety of formulae C and A, is t-butyloxycarbonylamino (—NHBOC).

One of ordinary skill in the art will recognize that there are a wide variety of reaction conditions that can be employed to reduce an amide, therefore, a wide variety of conditions are envisioned; see generally, March, (2001) and Larock (1999). In certain embodiments, the reduction step is performed by treating a compound of formula D with Red-Al [sodium bis(2-methoxyethoxy)aluminumhydride] or lithium aluminum hydride. In other embodiments, the reduction step is run in toluene, benzene, tetrahydrofuran, diethyl ether, tert-butyl methyl ether, or a mixture thereof. In certain embodiments, the reduction step is run at a temperature between about −40° C. and about 100° C. In other embodiments, the reduction step is run at a temperature between about 0° C. and 40° C. In still other embodiments the reduction is conducted in a manner substantially similar to that described in Gross, J. L. Tetrahetron Lett. 2003, 44, 8563; Mayweg, A. et al., U.S. patent application publication number U.S. 05250769 (2005); Devant, R. et al., International patent application publication number WO 05037817 (2005); Mitsuda, M. et al., International patent application publication number WO 03040382 (2003); Bokel, H. et al., International patent application publication number WO 02020507 (2002); or Bokel, H. et al., German patent application publication number DE 10120619 (2002).

Similarly, one of ordinary skill in the art will recognize that there are a wide variety of methods that can be employed to protect an amine, therefore, a wide variety of conditions are envisioned; see generally, Green (1999). In certain embodiments, the protection is performed in a manner substantially similar to that described in Van Lommen et al., International patent application publication number WO 9317017 (1993).

In step S-6, a CG¹ group is introduced at the open ortho position relative to the sp2-hybridized carbon bearing the chromane oxygen in formula C. The CG¹ group of formula A is a coupling group that facilitates transition metal-mediated Cₛₚ₂-Cₛₚ₂ coupling between the attached Cₛₚ₂ carbon and the Cₛₚ₂ carbon bearing a CG² coupling group in compounds of formula B, as shown in step S-7. Suitable coupling reactions are well known to one of ordinary skill in the art and typically involve one of the coupling groups being an electron-withdrawing group (e.g., Cl, Br, I, OTf, etc.), such that the resulting polar carbon-CG bond is susceptible to oxidative addition by an electron-rich metal (e.g., a low-valent palladium or nickel species), and the complementary coupling group being an electropositive group (e.g., boronic acids, boronic esters, boranes, stannanes, silyl species, zinc species, aluminum species, magnesium species, zirconium species, etc.), such that the carbon which bears the electropositive coupling group is susceptible to transfer to other electropositive species (e.g., a Pd^{II-IV} species or a Ni^{II-IV} species). Exemplary reactions and coupling groups include those described in Metal-Catalyzed Cross-Coupling Reactions, A. de Meijere and F. Diederich, Eds., 2^{nd} Edition, John Wiley & Sons, 2004; and Handbook of Organopalladium Chemistry for Organic Synthesis, Negishi, E., de Meijere, A. Editors, Wiley: New York, N.Y., 2002. In certain embodiments, CG¹ in compounds of formula A is a boronic acid, a boronic ester, or a borane. In other embodiments, CG¹ in compounds of formula A is a boronic ester. According to one aspect of the present invention, CG¹ in compounds of formula A is a boronic acid.

Reactions and reaction sequences that are used to promote the transformation depicted in step S-6 include initial directed orthometallation followed by treatment with suitable reagent to afford a compound of formula A. In certain embodiments, directed orthometallation is succeeded with treatment with a borate ester, which is optionally subsequently hydrolyzed to afford a boronic acid; see, e.g., Snieckus, V. Chem. Rev. 1990, 90, 879 and Schlosser, M. Angew. Chem. Int. Ed. 2005, 44, 376. Another exemplary sequence involves halogenation followed by a metallation/transmetallation sequence to afford a compound of formula A. In certain embodiments, halogenation and transmetallation is succeeded with treatment with a borate ester, which is optionally subsequently hydrolyzed to afford a boronic acid; see, generally, de Meijere (2004) and Snieckus (1990). According to one aspect of the present invention, a compound of formula C is first subjected to orthometallation to afford an intermediate arylmetal compound that is allowed to react with a borate ester to afford, following aqueous workup, a compound of formula A. In certain embodiments, the orthometallation is accomplished by treating a compound of formula C with an alkyl lithium reagent. In other embodiments the alkyllithium reagent employed is selected from tert-butyllithium, n-butyllithium, s-butyllithium, hexyllithium, and the like. In other embodiments the alkyllithium reagent employed is tert-butyllithium. In yet other embodiments, the reaction is run in tetrahydrofuran, diethyl ether, dimethoxyethane, tert-butyl methyl ether, or combinations thereof. In other embodiments, the lithiation reaction is run in tetrahydrofuran. In still other embodiments the reaction is run at a temperature between about 0° C. and about −90° C. In still other embodiments the reaction is run at a temperature between about −30° C. and about −50° C. In certain embodiments, the lithiation is run in the presence of one or more of N,N,N′,N′-tetramethylethylenediamine, or hexamethylphosphoric triamide. In other embodiments, the borate ester is triisopropylborate [B(OiPr)₃]. According to another aspect of the present invention, a compound of formula C is first brominated, then is subjected to halogen-metal exchange to afford an intermediate arylmetal compound that is allowed to react with a borate ester to afford, optionally following hydrolysis (by, e.g., treatment with aqueous hydrochloric acid, aqueous sulfuric acid, or the like) to the boronic acid, a compound of formula A.

In step S-7, a compound of formula A is coupled to a compound of formula B, via a Cₛₚ₂-Cₛₚ₂ coupling reaction between the carbon centers bearing complementary coupling groups CG¹ and CG² to provide a compound of formula II. Suitable coupling reactions and suitable coupling groups are as described above (see the description of embodiments for CG¹, above). In certain embodiments, CG² in compounds of formula B is Br, I, or OTf. According to one aspect of the present invention, CG² in compounds of formula B is Br.

In certain embodiments, the transformation is catalyzed by a palladium species. According to one aspect of the invention, the transformation is catalyzed by palladium tetrakis triphenylphosphine, Pd₂(dba)₃, or Pd(OAc)₂. In other embodiments, the palladium species is palladium tetrakis(triphenylphosphine).

In certain embodiments, the coupling reaction is run with dimethylacetamide, tetrahydrofuran, dimethoxyethane, toluene, dimethylformamide, N-methylpyrrolidine, or mixtures thereof, as solvent. In certain embodiments the coupling reaction is run with dimethylacetamide as solvent. According to another aspect of the present invention, the reaction is run in the presence of potassium phosphate or potassium carbonate. In other embodiments, the reaction is heated. According to one aspect of the invention, the reaction is heated to a temperature of about 100° C.

Each R² group of formulae B, II, and II•HX is independently Ph-, halogen, —CN, —R or —OR, wherein each R is independently hydrogen, C₁₋₆ aliphatic or C₁₋₆ haloaliphatic. Examples of suitable R² groups include methyl, ethyl, isopropyl, chloro, fluoro, methoxyl, trifluoromethyl, phenyl, cyano, ethoxyl, trifluoromethoxyl, and isopropoxyl. According to one aspect of the present invention, R² is chloro. According to another aspect of the present invention, at least one R² in ring B of compounds of formulae B, II, and II•HX, is located at one of the two ring positions that correspond to the positions ortho to CG² in formula B. According to yet another aspect of the present invention, an R² group is located at each of the two ring positions that correspond to the positions ortho to CG² in formula B. In certain embodiments, ring B is selected from those moieties depicted in Table 1, below (the [symbol] represents the point of attachment of ring B to CG² in compounds of formula B, or the point of attachment of ring B to ring A in compounds of formulae II and II•HX).

The numeral y of formulae B, II, and II•HX is 0-5. According to one aspect of the invention, y is 2.

TABLE 1

| | |
|---|---|
| | i |
| 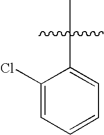 | |

| | |
|---|---|
| | ii |
| 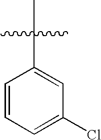 | |
| | iii |
| 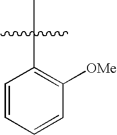 | |
| | iv |
| 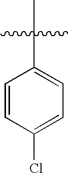 | |
| | v |
| 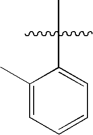 | |
| | vi |
| 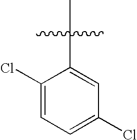 | |
| | vii |
| 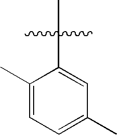 | |
| | viii |
| 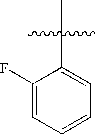 | |
| | ix |
| 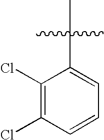 | |
| | x |
| 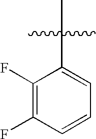 | |
| | xi |
| 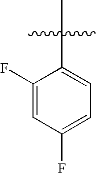 | |
| | xii |
| 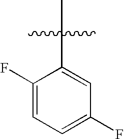 | |
| | xiii |
| 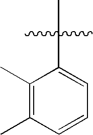 | |
| | xiv |
| 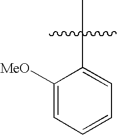 | |
| | xv |
| 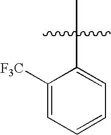 | |
| | xvi |
| 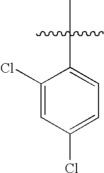 | |

| | |
|---|---|
| | xvii |
| 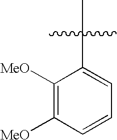 | |
| | xviii |
| 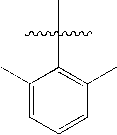 | |

| | |
|---|---|
| | xix |
| 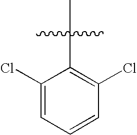 | |
| | xx |
| 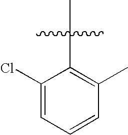 | |
| | xxi |
| 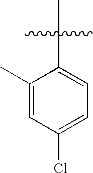 | |

| | |
|---|---|
| | xxii |
| 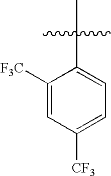 | |
| | xxiii |
| 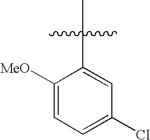 | |

| | |
|---|---|
| | xiv |
| 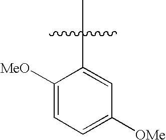 | |

| | |
|---|---|
| | xxv |
| 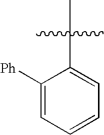 | |
| | xxvi |
| 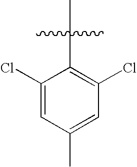 | |
| | xvii |
| 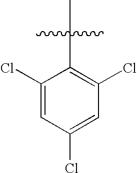 | |
| | xviii |
| 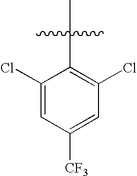 | |
| | xxix |
| 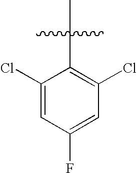 | |
| | xxx |
| 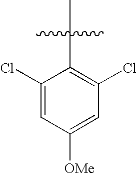 | |
| | xxxi |
| 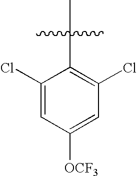 | |
| | xxxii |
| 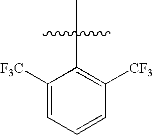 | |
| | xxxiii |
| 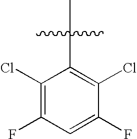 | |
| | xxxiv |
| 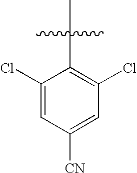 | |
| | xxxv |
| 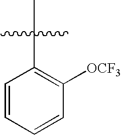 | |
| | xxxvi |
| 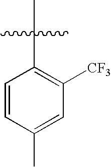 | |

| | |
|---|---|
| | xxxvii |
| 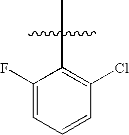 | |
| | xxxviii |
| 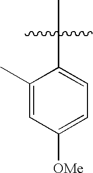 | |

| | |
|---|---|
| | xxxix |
| 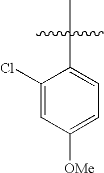 | |

| | |
|---|---|
| | xl |
| 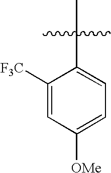 | |
| | xli |
| 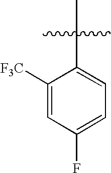 | |
| | xlii |
| 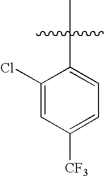 | |

| | |
|---|---|
| | xliii |
| 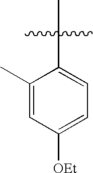 | |
| | xliv |
| 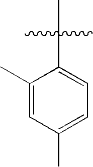 | |

| | |
|---|---|
| | xlv |
| 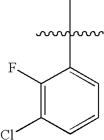 | |

| | |
|---|---|
| | xlvi |
| 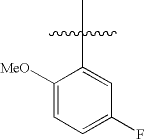 | |
| | xlvii |
| 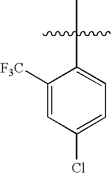 | |
| | xlviii |
| 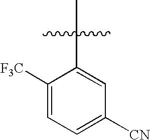 | |
| | xlix |
| 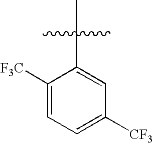 | |

| | |
|---|---|
| | xlx |
| 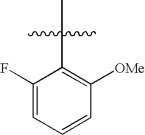 | |
| | li |
| 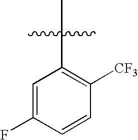 | |
| | lii |
| 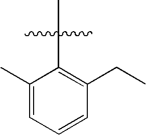 | |
| | liii |
| 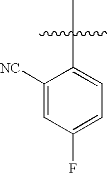 | |
| | liv |
| 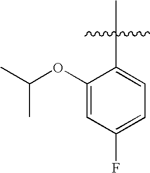 | |
| | lv |
| 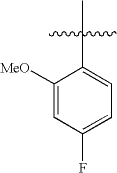 | |

Also in step S-7, following the coupling reaction, the amine protecting group is removed to provide compounds of formula II. One of ordinary skill in the art will recognize that there are a wide variety of methods that can be employed to deprotect an amine, therefore, a wide variety of conditions are envisioned; see generally, Green (1999). In certain embodiments where the —N(PG¹)(PG²) moiety of formulae C and A had been t-butyloxycarbonylamino (—NHBOC), the deprotection is conducted by treating the coupling product with a Brønsted acid selected from hydrochloric, sulfuric, trifluoroacetic, or trifluoromethanesulfonic acid. In certain embodiments, the deprotection step is run in water, methanol, ethanol, toluene, benzene, dichloromethane, dichloroethane, or chloroform.

One of ordinary skill in the art will appreciate that a compound of formula II, as prepared by the methods of the present invention, may be treated with a suitable Brønsted acid, HX, as depicted in step S-8, to form a salt thereof (represented by formula II•HX). Exemplary acids include hydrogen halides, carboxylic acids, sulfonic acids, sulfuric acid, and phosphoric acid. According to one aspect of the present invention, a compound of formula II is treated with HCl to form a compound of formula II•HX wherein X is Cl. In certain embodiments, where the acid is HCl, it is introduced into the medium containing the compound of formula II in gaseous form. In other embodiments, the acid is introduced into the medium containing the compound of formula II as a solution in methanol, ethanol, isopropanol, or water. In yet other embodiments, the acid is introduced into the medium containing the compound of formula II as a solution in isopropanol. In certain embodiments, the medium containing the compound of formula II is methanol. According to one aspect of the present invention, the deprotection step of step S-7 and the salt formation of step S-8 are conducted in a single step by employing the acid HX in the deprotection step.

One skilled in the art will appreciate that the enantiomeric excess of any of formulae E, D, C, A, II, and II•HX may be increased through a variety of means. Exemplary methods by which this may be accomplished include (a) the separation of enantiomers by chiral chromatographic methods, (b) selective crystallization of one enantiomer over the other, optionally by seeding a solution of the mixture of enantiomers with a crystal enriched in the desired enantiomer, (c) selective reaction of one enantiomer over the other with an enantioenriched chiral reaction partner, (d) selective reaction of one enantiomer over the other through chiral catalyst-promoted transformations (including enzymatic transformations), and (e) conversion of both enantiomers to corresponding diastereomers via either covalent or ionic bonding to a different enantiomerically enriched chiral species, followed by separation of the resulting diastereomers based upon their differing physical properties; for the above methods, see generally, Stereochemistry of Organic Compounds, E. L. Eliel and S. H. Silen, 1994; Enantiomers, Racemates and Resolutions, Jacques, et al. Wiley Interscience, New York, 1981; Wilen, S. H. et al., Tetrahedron 1977, 33, 2725; Tables of Resolving Agents and Optical Resolutions, Wilen, S. H. (E. L. Eliel, Ed.), Univ. of Notre Dame Press, Notre Dame, IN 1972. One of ordinary skill in the art will recognize that for preceding method (e), where both enantiomers of the compound of interest are converted by chemical means to a different chemical entity, that a subsequent step (or subsequent steps) will be necessary to reacquire the initial compounds.

It is further recognized that atropisomers of the present compounds may exit. The present invention thus encompasses atropisomeric forms of compounds of formulae II and II•HX as defined above, and in classes and subclasses described above and herein.

According to another aspect, the present invention provides an alternate method for preparing a compound of formula C from a compound of formula Q, and an alternate method for preparing a compound of formula II from a compound of formula L, as depicted in Scheme V below:

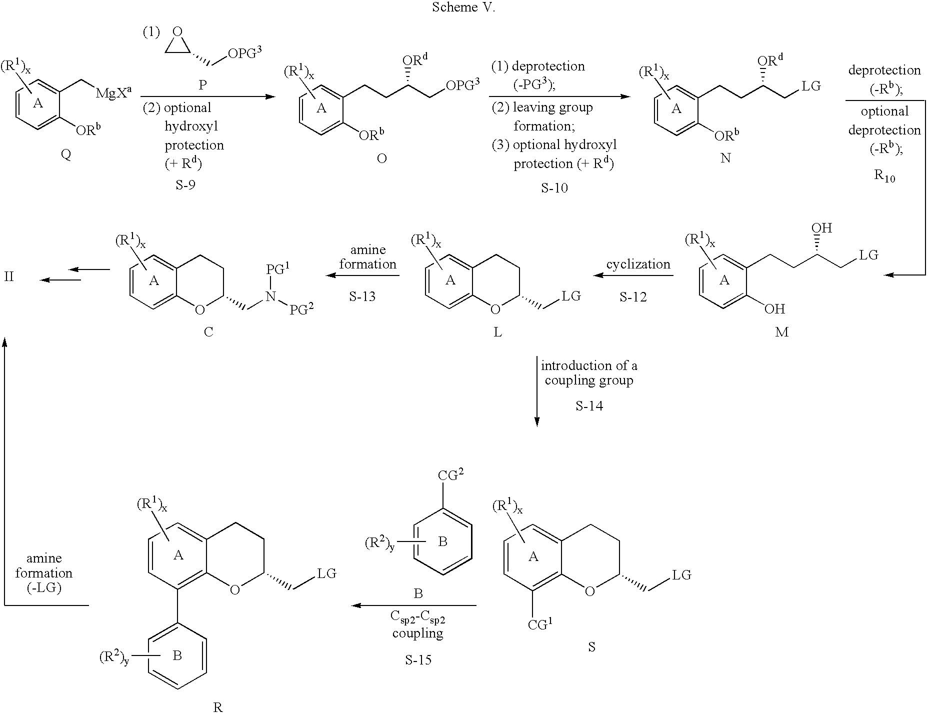

In step S-9, the Grignard adduct Q, wherein X^{a} is a halogen, and R^{b} is a suitable hydroxyl protecting group, is treated with a chiral non-racemic epoxide of the formula P, wherein PG³ is a suitable hydroxyl protecting group, to form a compound of formula O, wherein R^{d} is hydrogen. Addition of the epoxide can be optionally followed by hydroxyl group protection to form a compound of formula O, wherein R^{d} is a suitable hydroxyl protecting group. Protected hydroxyl groups (corresponding to —OPG³ of formulae O and P, OR^{b} of formulae Q, O, and N, and optionally, OR^{d} of formulae O and/or N) are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate(trimethylacetate), crotonate, 4-methoxy-crotonate, benzoate, p-benylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers. According to one aspect of the present invention, the PG³ group of formulae O and P is benzyl. According to another aspect of the present invention, group R^{b} of formulae Q, O and N is methyl. According to yet another aspect of the present invention, the group R^{d} of a compound of formula O is hydrogen.

For compounds of formulae Q and O, the R¹ and x are as defined above in embodiments and subembodiments for compounds of formula II. In certain embodiments, step S-9 includes formation of an organic cuprate. In some embodiments, the organic cuprate is CuCN, Li₂CuCl₄ or CuI. In other embodiments, step S-9 is performed in the presence of CuCN. In some embodiments, this step occurs at a temperature of about −15° C. to about −35° C. In yet other embodiments, this step occurs at a temperature of about −20° C. to about −25° C.

At step S-10, in the conversion of a compound of formula O to a compound of formula N, wherein R^{d} is hydrogen, the PG³ group of formula O is removed and the resulting hydroxyl group is either activated or replaced to provide a leaving group LG, wherein LG is a suitable leaving group that is subject to nucleophilic displacement. Alternatively, leaving group formation may be followed or accompanied by hydroxyl group protection to form a compound of formula N, wherein LG is a suitable leaving group and R^{d} is a suitable hydroxyl protecting group. Procedures for the removal of suitable hydroxyl protecting groups are well known in the art; see Green (1999). In certain embodiments, where PG³ is benzyl, PG³ is removed by treatment of a compound of formula O under reductive conditions or with conc HBr, acetic acid, or a mixture thereof. In other embodiments, wherein this step is conducted using HBr/acetic acid, the hydroxyl group of compound O wherein R^{d} is hydrogen, is optionally protected to form a compound of formula N, wherein LG is bromine, and R^{d} is acetyl.

A suitable “leaving group” that is “subject to nucleophilic displacement” is a chemical group that is readily displaced by a desired incoming nucleophilic chemical entity. Suitable leaving groups are well known in the art, e.g., see, Smith and March (2001). Such leaving groups include, but are not limited to, halogen, alkoxy, sulphonyloxy, optionally substituted alkylsulphonyloxy, optionally substituted alkenylsulfonyloxy, optionally substituted arylsulfonyloxy, and diazonium moieties. For the above mentioned “optionally substituted” moieties, the moieties may be optionally substituted with C₁₋₄ aliphatic, fluoro-substituted C₁₋₄ aliphatic, halogen, or nitro. Examples of suitable leaving groups include chloro, iodo, bromo, fluoro, methanesulfonyloxy (mesyloxy), tosyloxy, triflyloxy, nitro-phenylsulfonyloxy (nosyloxy), and bromo-phenylsulfonyloxy (brosyloxy). According to one aspect of the present invention, LG in compounds of formula N is toluenesulfonyloxy (tosyloxy). According to another aspect of the invention, the —OPG³ group is converted to a bromo (—Br) group. In certain embodiments, LG is bromine. In other embodiments, LG is bromine and R^{d} is acetyl.

Although Scheme V above depicts the deprotection of PG³ and the hydroxyl group activation to form the group LG as a single step, one of ordinary skill in the art will recognize that, depending on the choice of PG³, the deprotection and activation steps may also be performed in a stepwise fashion. The present invention contemplates such an alternate method.

At step S-11, the R^{b} hydroxyl protecting group of formula N is removed. As discussed above, procedures for the removal of suitable hydroxyl protecting groups are well known in the art; see Green (1999) and will depend upon the specific protecting group present in a compound of formula N. In certain embodiments, the R^{b} protecting group is a group that is deprotected using difference conditions as for the removal of PG³ thus facilitating the introduction of the LG group of formula N. In certain embodiments, the R^{b} group is alkyl. In other embodiments, R^{b} is methyl.

Cyclization of a compound of formula M to form a compound of formula L is depicted at step S-12 above. One of ordinary skill in the art will appreciate that various methods are known for such a cyclization. In certain embodiments, the cyclization is achieved by dehydration reaction. Dehydration reactions are well known to one of ordinary sill in the art. In other embodiments, the cyclization is achieved by Mitsunobu reaction. The Mitsunobu reaction is a mild method for achieving dehydration using azodicarboxylic esters/amides and triphenylphosphine (TPP) or phosphite. In addition, other azo compounds have been developed as alternatives to azodicarboxylic esters such as DIAD. These include dibenzyl azodicarboxylate (DBAD), N,N,N′,N′-tetramethylazodicarbonamide (TMAD), and dipiperidyl azodicarboxylate (DPAD).

At step S-13, a protected amine moiety is introduced via displacement of the LG group of a compound of formula L to afford a compound of formula C. In compounds of formulae C (Scheme V) and A (Scheme I), PG¹ and PG² are amino protecting groups. Alternatively, at step S-13, the LG group of a compound of formula L can be displaced with an amine, and then protected, to afford a compound of formula C. Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, and 2,2,5,5-tetramethyl-[1,2,5]azadisilolidine and the like.

Notwithstanding the definition above, one of either PG¹ or PG² in compounds of formulae C and A may be hydrogen. Also notwithstanding the definitions above, the —N(PG¹)(PG²) moiety of formulae C and A may be azido. According to one aspect of the invention, the —N(PG¹)(PG²) moiety of formulae C and A, is phthalimido. According to another aspect of the invention, at step S-13, a compound of formula L is treated with potassium phthalimide to generate compounds of formula C in which the —N(PG¹)(PG²) moiety is phthalimido. In other embodiments, the LG group of a compound of formula L can be displaced with azide. In yet other embodiments, the LG group of a compound of formula L can be displaced with azide, reduced to an amine, and then protected, to afford a compound of formula C.

In certain embodiments, step S-13 is performed with heating. In other embodiments, the reaction is conducted at a temperature that is between about 40° C. and about 110° C. In other embodiments, the reaction is run at about 90° C.

In certain embodiments, step S-13 is conducted in the presence of a polar aprotic solvent. Exemplary polar aprotic solvents include dimethylformamide (DMF), N-methylpyrrolidine (NMP), dimethylacetamide (DMA), dioxane, tetrahydrofuran (THF), and dimethylsulfoxide (DMSO). In certain embodiments, the reaction is conducted in dimethylformamide (DMF), N-methylpyrrolidine (NMP), or dimethylacetamide (DMA). In other embodiments, the reaction is conducted in DMF.

Compounds of formula L are transformed to compounds of formula C via step S-13, which in turn may be transformed to compounds of formulae II and II•HX according to steps S-6, S-7, and S-8 as described in detail above and herein with respect to Scheme I and Scheme V. One of ordinary skill in the art, however, will appreciate that there are alternative ways of making a compound of formula II from a compound of formula L. For instance, as depicted in Scheme V, a coupling group may be introduced to form a compound of formula S, which may be coupled to a compound of formula B to form a compound of formula R, which may be aminated to afford a compound of formula II. The reaction steps corresponding to the above transformations S-14, S-15 and S-16 have been described in detail herein in reference to corresponding reaction steps (for example, Scheme I above).

As used herein, the term “aliphatic” or “aliphatic group”, as used herein, means a straight-chain (i.e., unbranched) or branched, hydrocarbon chain that is completely saturated or that contains one or more units of unsaturation, or a monocyclic hydrocarbon that is completely saturated or that contains one or more units of unsaturation, but which is not aromatic (also referred to herein as “carbocycle” or “cycloaliphatic”), that has a single point of attachment to the rest of the molecule. In certain embodiments, aliphatic groups contain 1-6 carbon atoms, and in yet other embodiments, aliphatic groups contain 1-3 carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Such groups include cycloalkyl, cycloalkenyl, and cycloalkynyl groups. Suitable aliphatic groups include, but are not limited to, linear or branched, alkyl, alkenyl, alkynyl groups and hybrids thereof such as (cycloalkyl)alkyl, (cycloalkenyl)alkyl or (cycloalkyl)alkenyl.

The term “unsaturated,” as used herein, means that a moiety has one or more units of unsaturation.

The term “alkyl,” as used herein, refers to a hydrocarbon chain having up to 6 carbon atoms. The term “alkyl” includes, but is not limited to, straight and branched chains such as methyl, ethyl, n-propyl, isopropyl, n-butyl, isobutyl, sec-butyl, t-butyl, n-pentyl, iso-pentyl, 1-methyl-butyl, 2-methyl-butyl, n-hexyl, 1-methyl-pentyl, 2-methyl-pentyl, 3-methyl-pentyl, or 4methyl-pentyl.

The terms “halogen” or “halo,” as used herein, refer to a chloro (—Cl), bromo (—Br), fluoro (—F) or iodo (—I) atom.

The term “haloaliphatic,” as used herein, refers to an aliphatic group, as defined herein, that has one or more halogen substituents. In certain embodiment, every hydrogen atom on said aliphatic group is replaced by a halogen atom. Such haloaliphatic groups include —CF₃.

The term “fluoroaliphatic,” as used herein, an aliphatic group, as defined herein, that has one or more fluorine substituents. In a certain embodiment, a fluoroaliphatic group is a fluoroalkyl group.

The term “fluoroalkyl,” as used herein, refers to an alkyl group, as defined herein, that has one or more fluorine substituents. In certain embodiment, every hydrogen atom on said alkyl group is replaced by a fluorine atom.

The term “Ph,” as used herein, refers to a phenyl group.

The term “alkenyl,” as used herein refers to an aliphatic straight or branched hydrocarbon chain having 2 to 8 carbon atoms that may contain 1 to 3 double bonds. Examples of alkenyl groups include vinyl, prop-1-enyl, allyl, methallyl, but-1-enyl, but-2-enyl, but-3-enyl, or 3,3-dimethylbut-1-enyl. In some embodiments, the alkenyl is preferably a branched alkenyl of 3 to 8 carbon atoms.

The term “pharmaceutically acceptable salts” or “pharmaceutically acceptable salt” includes acid addition salts, that is salts derived from treating a compound of formula II with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. Where a compound of formula I contains a substituent with acidic properties, the term also includes salts derived from bases, for example, sodium salts. In certain embodiments, the present invention provides the hydrochloride salt of a compound of formula II.

According to another aspect, the present invention provides a method for preparing a compound of formula II•HX: 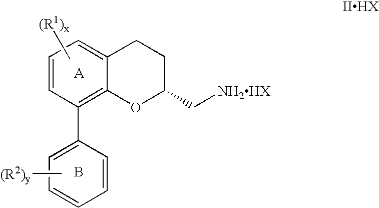
wherein:

x is 0-3;

y is 0-5;

each R¹ is independently —R, —CN, halogen or —OR;

each R is independently hydrogen, C₁₋₆ aliphatic or C₁₋₆ haloaliphatic;

each R² is independently —Ph, halogen, —CN, —R or —OR; and

X is the anion of a suitable acid,

comprising the steps of:

(a) providing a compound of formula II: 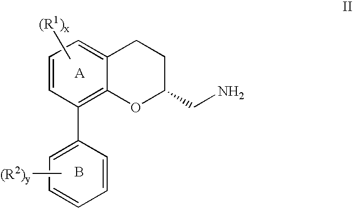
   wherein:

x is 0-3;

y is 0-5;

each R¹ is independently —R, —CN, halogen or —OR;

each R is independently hydrogen, C₁₋₆ aliphatic or C₁₋₆ haloaliphatic; and

each R² is independently -Ph, halogen, —CN, —R or —OR,

and

(b) reacting said compound of formula II with suitable acid of formula HX to form a compound of formula II•X.

As defined above, in compounds of formulae II and II•HX, x is 0-3, y is 0-5, each R¹ is independently —R, —CN, halogen or —OR, each R is independently hydrogen, C₁₋₆ aliphatic or C₁₋₆ haloaliphatic, and each R² is independently R, -Ph, —CN, halogen, or —OR. In certain embodiments, x is 0-2. In other embodiments, x is 0. In certain embodiments, y is 2-3. In other embodiments, y is 2. In certain embodiments, R¹ is —F or —Cl. In other embodiments, R¹ is fluoro. In certain embodiments, R² is —F, —Cl, or C₁₋₃ aliphatic. In other embodiments, R² is chloro. In certain embodiments, ring A is substituted with an R¹ group at the open meta position relative to the carbon bearing ring B. In other embodiments, Ring B is substituted with at least one R² group at a position ortho to the carbon bearing Ring A. In yet other embodiments, Ring B is substituted at each position ortho to the carbon bearing ring A with an R² group.

In certain embodiments, the compound of formula II is selected from those depicted in Table 2, below.

TABLE 2

| | |
|---|---|
| | II-1 |
| 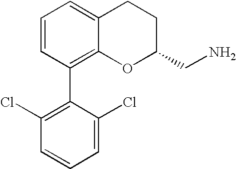 | |
| | II-2 |
| 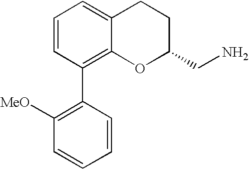 | |
| | II-3 |
| 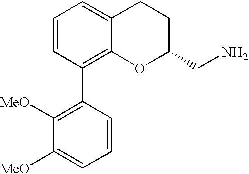 | |
| | II-4 |
| 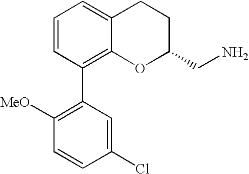 | |
| | II-5 |
| 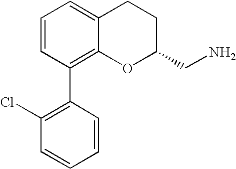 | |
| | II-6 |
| 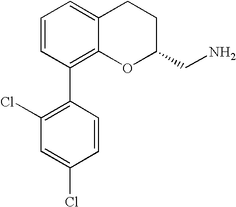 | |
| | II-7 |
| 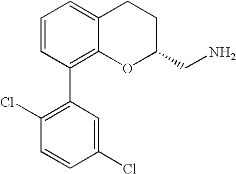 | |
| | II-8 |
| 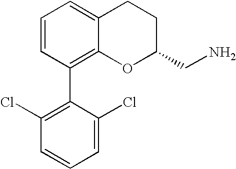 | |
| | II-9 |
| 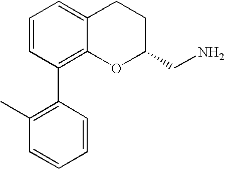 | |
| | II-10 |
| 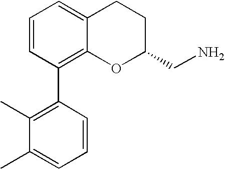 | |
| | II-11 |
| 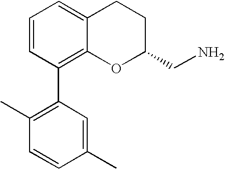 | |
| | II-12 |
| 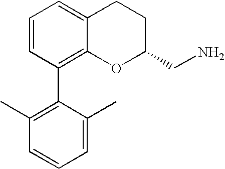 | |
| | II-13 |
| 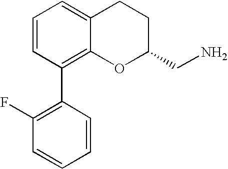 | |
| | II-14 |
| 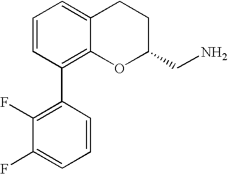 | |
| | II-15 |
| 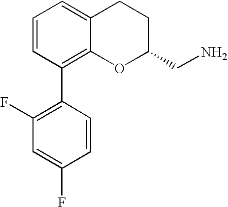 | |
| | II-16 |
| 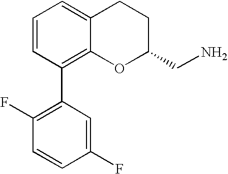 | |
| | II-17 |
| 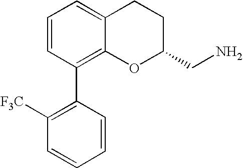 | |
| | II-18 |
| 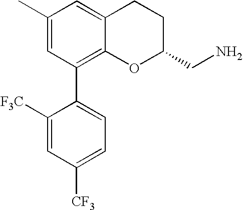 | |
| | II-19 |
| 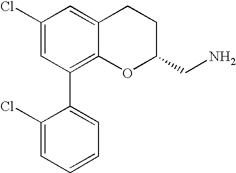 | |
| | II-20 |
| 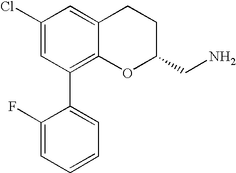 | |
| | II-21 |
| 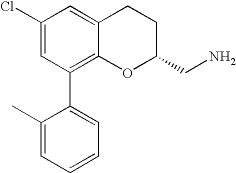 | |
| | II-22 |
| 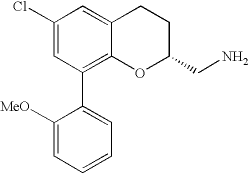 | |
| | II-23 |
| 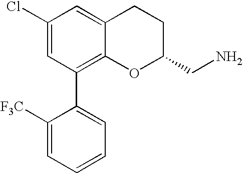 | |
| | II-24 |
| 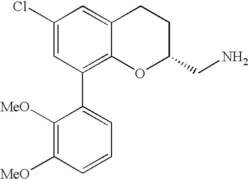 | |
| | II-25 |
| 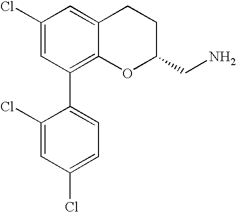 | |
| | II-26 |
| 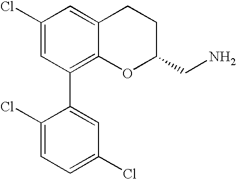 | |
| | II-27 |
| 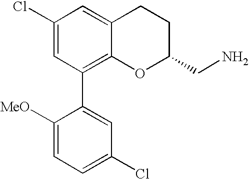 | |
| | II-28 |
| 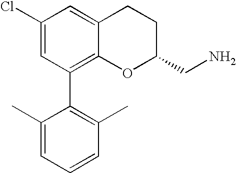 | |
| | II-29 |
| 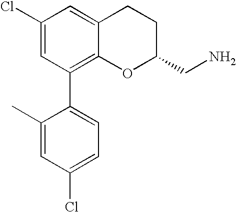 | |
| | II-30 |
| 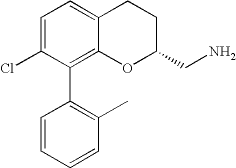 | |
| | II-31 |
| 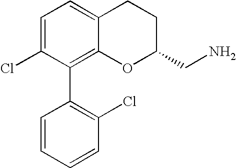 | |
| | II-32 |
| 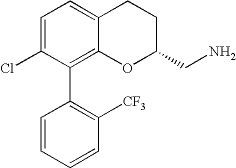 | |
| | II-33 |
| 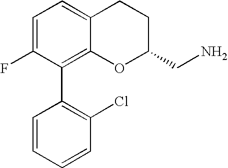 | |
| | II-34 |
| 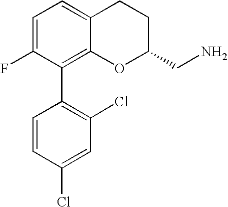 | |
| | II-35 |
| 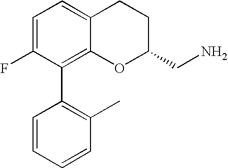 | |
| | II-36 |
| 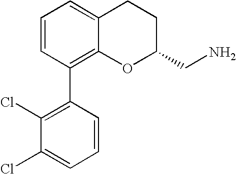 | |
| | II-37 |
| 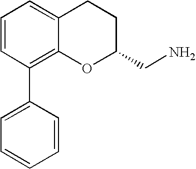 | |
| | II-38 |
| 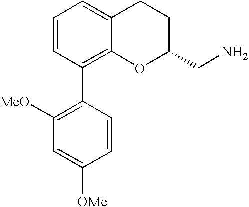 | |
| | II-39 |
| 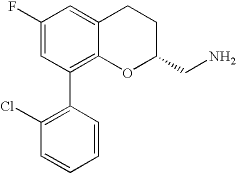 | |
| | II-40 |
| 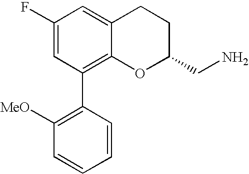 | |
| | II-41 |
| 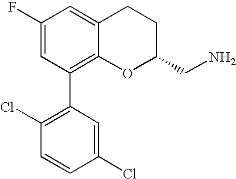 | |
| | II-42 |
| 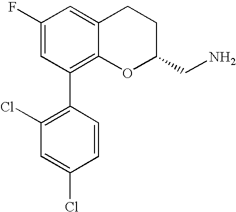 | |
| | II-43 |
| 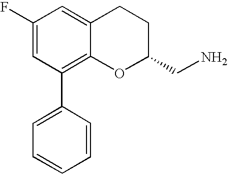 | |
| | II-44 |
| 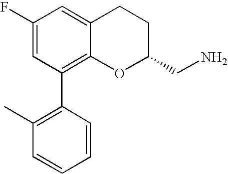 | |
| | II-45 |
| 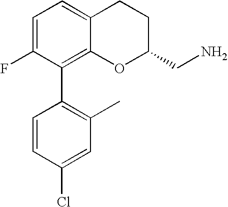 | |
| | II-46 |
| 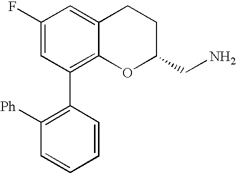 | |
| | II-47 |
| 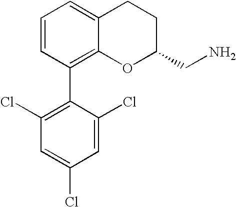 | |
| | II-48 |
| 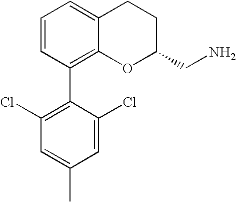 | |
| | II-49 |
| 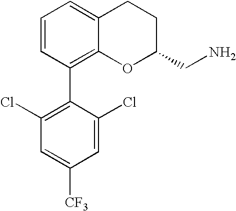 | |
| | II-50 |
| 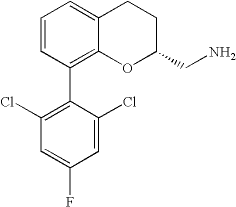 | |
| | II-51 |
| 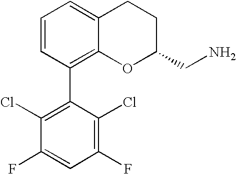 | |
| | II-52 |
| 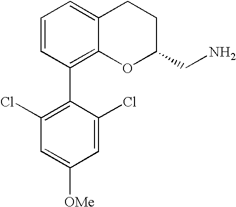 | |
| | II-53 |
| 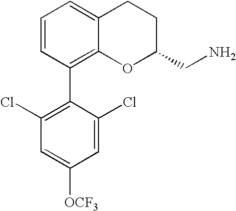 | |
| | II-54 |
| 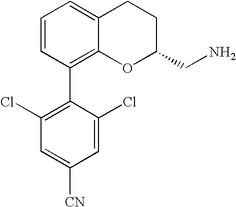 | |
| | II-55 |
| 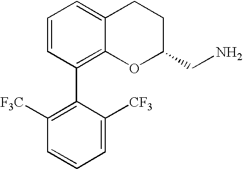 | |
| | II-56 |
| 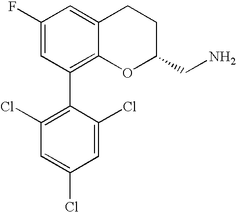 | |
| | II-57 |
| 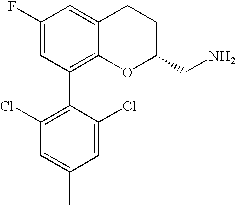 | |
| | II-58 |
| 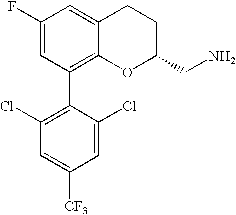 | |
| | II-59 |
| 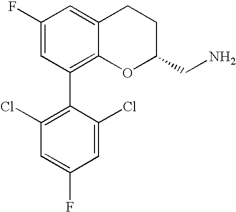 | |
| | II-60 |
| 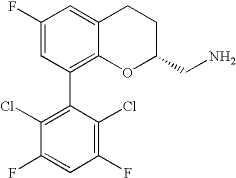 | |
| | II-61 |
| 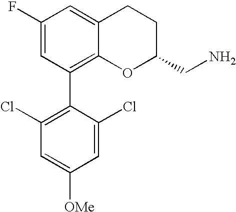 | |
| | II-62 |
| 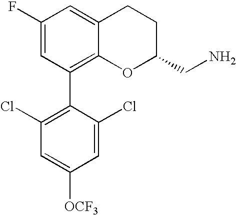 | |
| | II-63 |
| 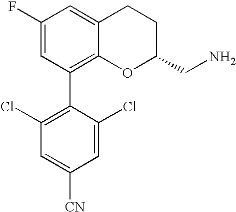 | |
| | II-64 |
| 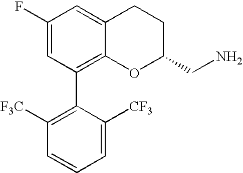 | |
| | II-65 |
| 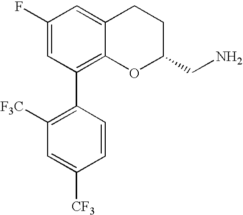 | |
| | II-66 |
| 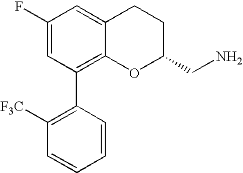 | |
| | II-67 |
| 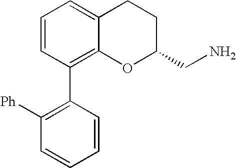 | |
| | II-68 |
| 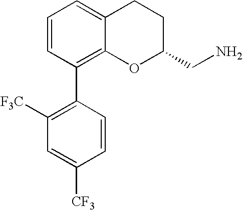 | |
| | II-69 |
| 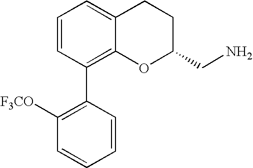 | |
| | II-70 |
| 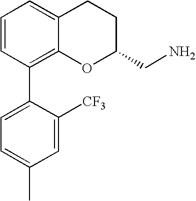 | |
| | II-71 |
| 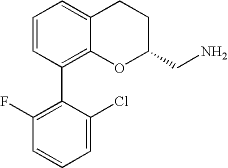 | |
| | II-72 |
| 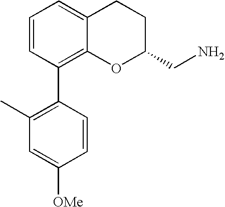 | |
| | II-73 |
| 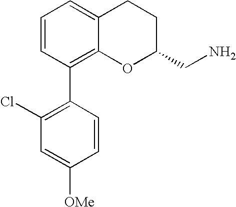 | |
| | II-74 |
| 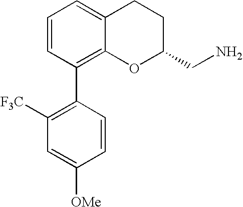 | |
| | II-75 |
| 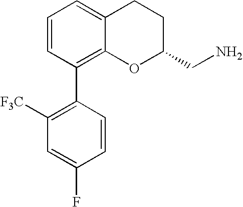 | |
| | II-76 |
| 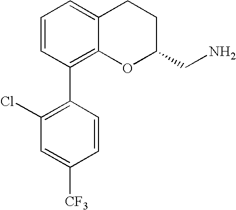 | |
| | II-77 |
| 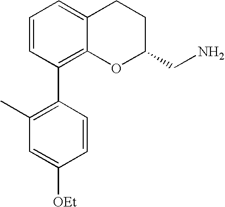 | |
| | II-78 |
| 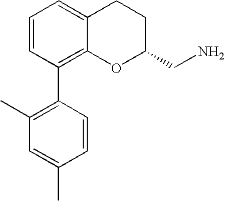 | |
| | II-79 |
| 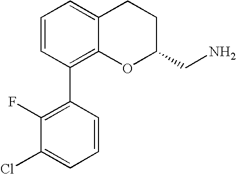 | |
| | II-80 |
| 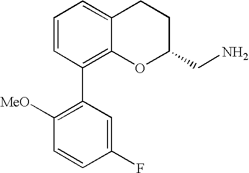 | |
| | II-81 |
| 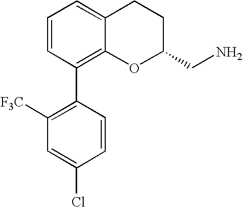 | |
| | II-82 |
| 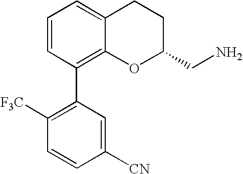 | |
| | II-83 |
| 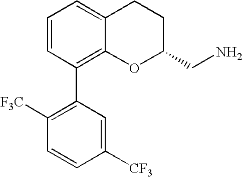 | |
| | II-84 |
| 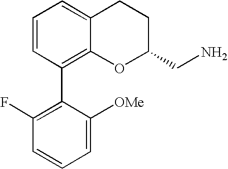 | |
| | II-85 |
| 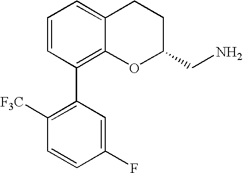 | |
| | II-86 |
| 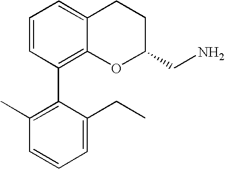 | |
| | II-87 |
| 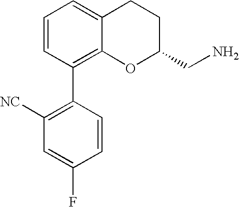 | |
| | II-88 |
| 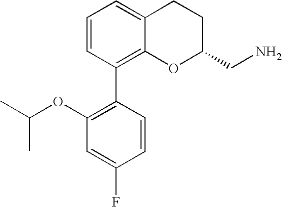 | |
| | II-89 |
| 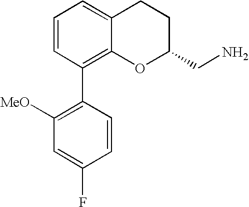 | |
| | II-90 |
| 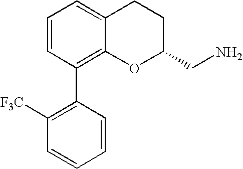 | |
| | II-91 |
| 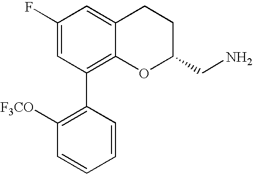 | |
| | II-92 |
| 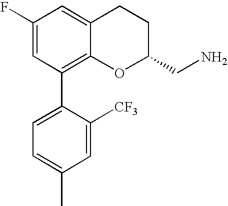 | |
| | II-93 |
| 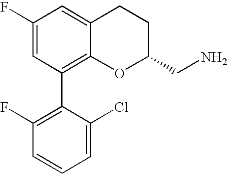 | |
| | II-94 |
| 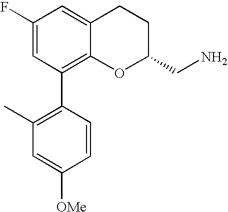 | |
| | II-95 |
| 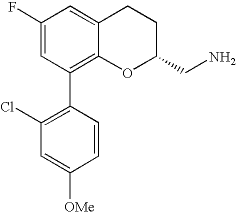 | |
| | II-96 |
| 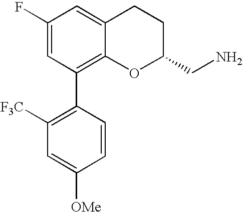 | |
| | II-97 |
| 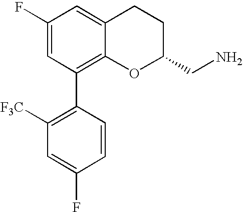 | |
| | II-98 |
| 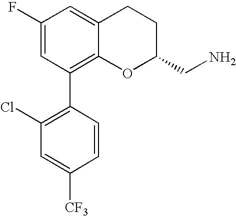 | |
| | II-99 |
| 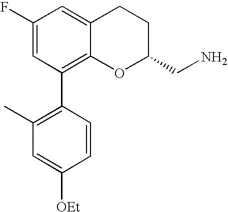 | |

| | |
|---|---|
| | II-100 |
| 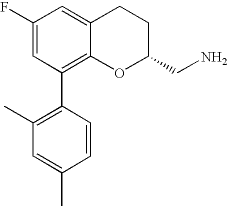 | |
| | II-101 |
| 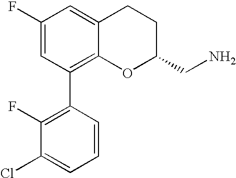 | |
| | II-102 |
| 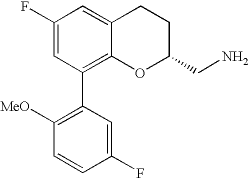 | |
| | II-103 |
| 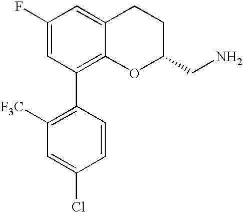 | |
| | II-104 |
| 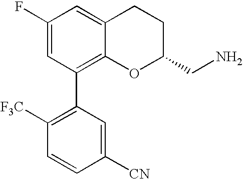 | |
| | II-105 |
| 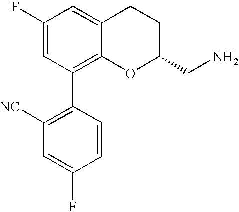 | |
| | II-106 |
| 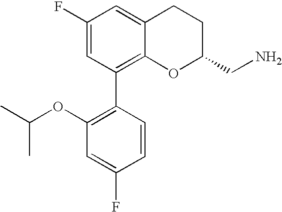 | |
| | II-107 |
| 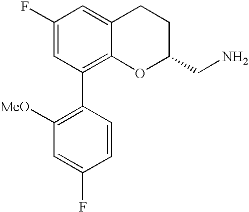 | |

In other embodiments, the compound of formula II is selected from II-1, II-8, and II-47. In yet another embodiment, the compound of formula II is II-1.

As defined above, HX in the reaction step above and in compounds of formula II•HX is a suitable Brønsted acid. Exemplary acids include hydrogen halides, carboxylic acids, sulfonic acids, sulfuric acid, and phosphoric acid. According to one aspect of the present invention, a compound of formula II is treated with HCl to form a compound of formula II•HX wherein X is Cl. In certain embodiments, where the acid is HCl, the acid is introduced into the medium containing the compound of formula II in gaseous form. In other embodiments, the acid is introduced into the medium containing the compound of formula II as a solution in methanol, ethanol, isopropanol, or water. In yet other embodiments, the acid is introduced into the medium containing the compound of formula II as a solution in isopropanol. In certain embodiments, the medium containing the compound of formula II is methanol.

In certain embodiments, the compound of formula II•HX is selected from the group of compounds formed by combining those compounds of formula II depicted in Table 2 with a suitable Brønsted acid. In other embodiments, the compound of formula II•HX is selected from those salts formed by combining compound II-1 with a suitable Brønsted acid. In yet another embodiment, the compound of formula II•HX is the HCl salt of compound II-1.

In certain embodiments, the compound of formula II•HX is isolated by crystallization. In other embodiments, this crystallization step serves as the only isolation or purification step for compounds of this formula. In still other embodiments, the crystallization is optionally repeated until the compound of formula II•HX is of desired purity. In yet other embodiments, this crystallization increases the enantiomeric excess of the crystalline product, and is optionally conducted by seeding the solution of the enantiomers of formula II•HX with one or more crystals of the same that is enriched in the desired enantiomeric form.

According to another embodiment, the present invention provides a method for preparing a compound of formula II: 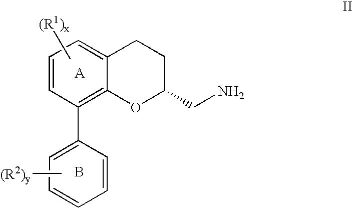
wherein:
x is 0-3;
y is 0-5;
each R¹ is independently —R, —CN, halogen or —OR;
each R is independently hydrogen, C₁₋₆ aliphatic or C₁₋₆ haloaliphatic; and
each R² is independently -Ph, halogen, —CN, —R or —OR,
   comprising the steps of:
(a) providing a compound of formula A: 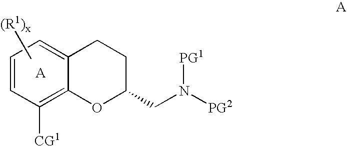
   wherein:
   x is 0-3;
   each R¹ is independently —R, —CN, halogen or —OR;
   each R is independently hydrogen, C₁₋₆ aliphatic or C₁₋₆ haloaliphatic;
   PG¹ and PG² are each independently hydrogen or a suitable amino protecting group; and
   CG¹ is a coupling group that facilitates transition metal-mediated Cₛₚ₂-Cₛₚ₂ coupling between the attached Cₛₚ₂ carbon and a Cₛₚ₂ carbon bearing a CG² coupling group,
(b) coupling said compound of formula A with a compound of formula B: 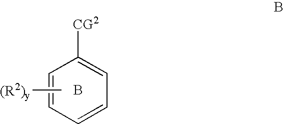
   wherein:
   y is 0-5;
   each R is independently hydrogen, C₁₋₆ aliphatic or C₁₋₆ haloaliphatic;
   each R² is independently -Ph, halogen, —CN, —R or —OR; and
   CG² is a coupling group that facilitates transition metal-mediated Cₛₚ₂-Cₛₚ₂ coupling between the attached Cₛₚ₂ carbon and a Cₛₚ₂ carbon bearing a CG¹ coupling group;
   in the presence of a suitable transition metal,
      and
(c) deprotecting the protected amine moiety of the coupling product to form a compound of formula II.

For compounds of formula A, each of x and R¹ are as defined above in embodiments and subembodiments for compounds of formula II and II•HX. As defined above, the PG¹ and PG² groups of compounds of formula A are each independently hydrogen or a suitable amino protecting group. Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, allyloxycarbonylamino (—NHAlloc), benzyloxocarbonylamino (—NHCBz), allylamino, benzylamino (—NHBn), formamido, and the like. Suitable di-protected amines include amines that are substituted with two substituents independently selected from those described above as mono-protected amines, and further include Suitable di-protected amines also include pyrroles and the like. In certain embodiments, one of either PG¹ or PG² or both in compounds of formulae C and A may be hydrogen. The amino group can also be masked as an azido group —N₃. According to one aspect of the invention, the —N(PG¹)(PG²) moiety of formulae C and A, is t-butyloxycarbonylamino (—NHBOC).

As defined above, the CG¹ group of compounds of formula A is a coupling group that facilitates transition metal-mediated Cₛₚ₂-Cₛₚ₂ coupling between the attached Cₛₚ₂ carbon and a Cₛₚ₂ carbon bearing a CG² coupling group. Similarly, as defined above, for compounds of formula B, CG² is a coupling group that facilitates transition metal-mediated Cₛₚ₂-Cₛₚ₂ coupling between the attached Cₛₚ₂ carbon and a Cₛₚ₂ carbon bearing a CG¹ coupling group.

For compounds of formula B, each of y and R² are as defined above in embodiments and subembodiments for compounds of formula II and II•HX.

In this coupling step, a compound of formula A is coupled to a compound of formula B, via a Cₛₚ₂-Cₛₚ₂ coupling reaction between the carbon centers bearing complementary coupling groups CG¹ and CG² to provide a compound of formula II. Suitable coupling reactions are well known to one of ordinary skill in the art and typically involve one of CG¹ or CG² being an electron-withdrawing group (e.g., Cl, Br, I, OTf, etc.), such that the resulting polar carbon-CG bond is susceptible to oxidative addition by an electron-rich metal (e.g., a low-valent palladium or nickel species), and the complementary coupling group being an electropositive group (e.g., boronic acids, boronic esters, boranes, stannanes, silyl species, zinc species, aluminum species, magnesium species, zirconium species, etc.), such that the carbon which bears the electropositive coupling group is susceptible to transfer to other electropositive species (e.g., a Pd^{II-IV} species or a Ni^{II-IV} species). Exemplary reactions include those described in Metal-Catalyzed Cross-Coupling Reactions, A. de Meijere and F. Diederich, Eds., 2^{nd} Edition, John Wiley & Sons, 2004; and Handbook of Organopalladium Chemistry for Organic Synthesis, Negishi, E., de Meijere, A. Editors, Wiley: New York, N.Y., 2002. In certain embodiments, CG¹ in compounds of formula A is a boronic acid, a boronic ester, or a borane. In other embodiments, CG¹ in compounds of formula A is a boronic ester. According to one aspect of the present invention, CG¹ in compounds of formula A is a boronic acid. According to one aspect of the present invention, the compound of formula A is 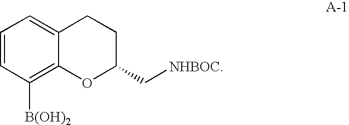
In certain embodiments, CG² in compounds of formula B is Br, I, or OTf. According to one aspect of the present invention, CG² in compounds of formula B is Br. According to another aspect of the present invention, the compound of formula B is

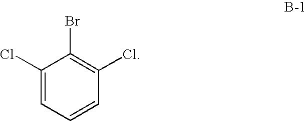

In certain embodiments, the coupling reaction is run with dimethylacetamide, tetrahydrofuran, dimethoxyethane, toluene, dimethylformamide, N-methylpyrrolidine, or mixtures thereof, as solvent. In certain embodiments the coupling reaction is run with dimethylacetamide as solvent.

According to another aspect of the present invention, the reaction is run in the presence of potassium phosphate or potassium carbonate.

In other embodiments, the reaction is heated. According to one aspect of the invention, the reaction is heated to a temperature of about 100° C.

According to another embodiment, the present invention provides a method for preparing a compound of formula A: 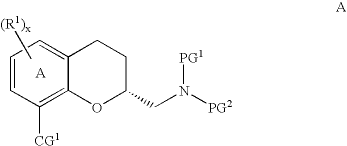
wherein:
x is 0-3;
each R¹ is independently —R, —CN, halogen or —OR;
each R is independently hydrogen, C₁₋₆ aliphatic or C₁₋₆ haloaliphatic;
PG¹ and PG² are each independently hydrogen or a suitable amino protecting group; and
CG¹ is a coupling group that facilitates transition metal-mediated Cₛₚ₂-Cₛₚ₂ coupling between the attached Cₛₚ₂ carbon and a Cₛₚ₂ carbon bearing a CG² coupling group, comprising the steps of:
(a) providing a compound of formula C: 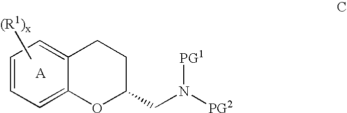
   wherein:
   x is 0-3;
   each R¹ is independently —R, -Ph, —CN, halogen, or —OR;
   each R is independently hydrogen, C₁₋₃ aliphatic or C₁₋₃ fluoroaliphatic; and
   PG¹ and PG² are each independently hydrogen or a suitable amino protecting group,
      and
(b) introducing a CG¹ group at the open ortho position relative to the sp2-hybridized carbon bearing the chromane oxygen in formula C to afford a compound of formula A.

For compounds of formula C, each of x and R¹ are as defined above in embodiments and subembodiments for compounds of formula II and II•HX, and each of PG¹ and PG² are as defined above in embodiments and subembodiments for compounds of formula A.

In this step, a CG¹ group is introduced at the open ortho position relative to the sp2-hybridized carbon bearing the chromane oxygen in formula C. Reactions and reaction sequences that are used to promote this transformation include initial directed orthometallation followed by treatment with suitable reagent to afford a compound of formula A. In certain embodiments, directed orthometallation is succeeded with treatment with a borate ester, which is optionally subsequently hydrolyzed to afford a boronic acid; see, e.g., Snieckus (1990) and Schlosser (2005). Another exemplary sequence involves halogenation followed by a metallation/transmetallation sequence to afford a compound of formula A. In certain embodiments, halogenation and transmetallation is succeeded with treatment with a borate ester, which is optionally subsequently hydrolyzed to afford a boronic acid; see, generally, de Meijere (2004) and Snieckus (1990).

According to one aspect of the present invention, a compound of formula C is first subjected to orthometallation to afford an intermediate arylmetal compound that is allowed to react with a borate ester to afford, following aqueous workup, a compound of formula A. In certain embodiments, the orthometallation is accomplished by treating a compound of formula C with an alkyl lithium reagent. In other embodiments the alkyllithium reagent employed is selected from tert-butyllithium, n-butyllithium, s-butyllithium, hexyllithium, and the like. In other embodiments the alkyllithium reagent employed is tert-butyllithium.

In certain embodiments, the reaction is run in tetrahydrofuran, diethyl ether, dimethoxyethane, tert-butyl methyl ether, or combinations thereof. In other embodiments, the lithiation reaction is run in tetrahydrofuran.

In still other embodiments the reaction is run at a temperature between about 0° C. and about −90° C. In still other embodiments the reaction is run at a temperature between about −30° C. and about −50° C.

In certain embodiments, the lithiation is run in the presence of one or more of N,N,N′,N′-tetramethylethylenediamine, or hexamethylphosphoric triamide.

In other embodiments, the borate ester is triisopropylborate [B(OiPr)₃]. According to another aspect of the present invention, a compound of formula C is first brominated, then is subjected to halogen-metal exchange to afford an intermediate arylmetal compound that is allowed to react with a borate ester to afford, optionally following hydrolysis (by, e.g., treatment with aqueous hydrochloric acid, aqueous sulfuric acid, or the like) to the boronic acid, a compound of formula A.

According to one aspect of the present invention, the compound of formula C is

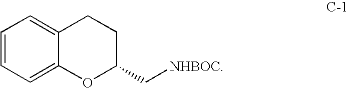

According to another embodiment, the present invention provides a method for preparing a compound of formula C: 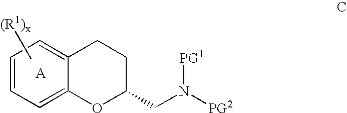
wherein:

x is 0-3;

each R¹ is independently —R, —CN, halogen or —OR;

each R is independently hydrogen, C₁₋₆ aliphatic or C₁₋₆ haloaliphatic; and

PG¹ and PG² are each independently hydrogen or a suitable amino protecting group, comprising the steps of:
(a) providing a compound of formula D: 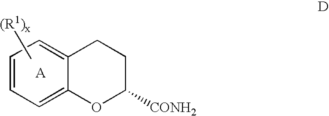
   wherein:

x is 0-3;

each R¹ is independently —R, —CN, halogen or —OR; and

each R is independently hydrogen, C₁₋₆ aliphatic or C₁₋₆ haloaliphatic;
(b) reducing the amide moiety in the compound of formula D to the amine;
   and
(c) optionally protecting the amine moiety resulting from the reduction of the amide moiety in the compound of formula D with a suitable amine protecting group to afford a compound of formula C.

For compounds of formula D, each of x and R¹ are as defined above in embodiments and subembodiments for compounds of formula II and II•HX.

In this step, the amide moiety in compounds of formula D is reduced to an amine, and the resulting amine is optionally protected to afford compounds of formula C. One of ordinary skill in the art will recognize that there are a wide variety of reaction conditions that can be employed to reduce an amide, therefore, a wide variety of conditions are envisioned; see generally, March, (2001) and Larock (1999). In certain embodiments, the reduction step is performed by treating a compound of formula D with Red-Al [sodium bis(2-methoxyethoxy)aluminumhydride] or lithium aluminum hydride. In other embodiments, the reduction step is run in toluene, benzene, tetrahydrofuran, diethyl ether, tert-butyl methyl ether, or a mixture thereof. In certain embodiments, the reduction step is run at a temperature between about −40° C. and about 100° C. In other embodiments, the reduction step is run at a temperature between about 0° C. and about 40° C. In still other embodiments the reduction is conducted in a manner substantially similar to that described in Gross, J. L. Tetrahetron Lett. 2003, 44, 8563; Mayweg, A. et al., U.S. patent application publication number US 05250769 (2005); Devant, R. et al., International patent application publication number WO 05037817 (2005); Mitsuda, M. et al., International patent application publication number WO 03040382 (2003); Bokel, H. et al., International patent application publication number WO 02020507 (2002); or Bokel, H. et al., German patent application publication number DE 10120619 (2002).

Similarly, one of ordinary skill in the art will recognize that there are a wide variety of methods that can be employed to protect an amine, therefore, a wide variety of conditions are envisioned; see generally, Green (1999). In certain embodiments, the protection is performed substantially similar to that described in Van Lommen et al., International patent application publication number WO 9317017 (1993).

According to another embodiment, the present invention provides a method for preparing a compound of formula D: 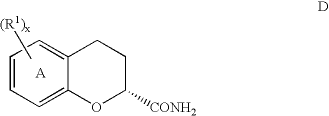
wherein:

x is 0-3;

each R¹ is independently —R, —CN, halogen or —OR; and

each R is independently hydrogen, C₁₋₆ aliphatic or C₁₋₆ haloaliphatic;

comprising the steps of:

(a) providing a compound of formula E: 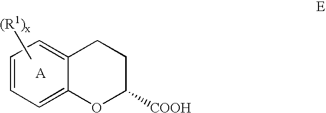
   wherein:

x is 0-3;

each R¹ is independently —R, —CN, halogen or —OR; and

each R is independently hydrogen, C₁₋₆ aliphatic or C ₁₋₆ haloaliphatic;

and

(b) converting the carboxylic acid moiety in the compound of formula E into an amide moiety to form a compound of formula D.

For compounds of formula E, each of x and R¹ are as defined above in embodiments and subembodiments for compounds of formula II and II•HX.

In this amidation step, a compound of formula E is amidated to afford a compound of formula D. One of ordinary skill in the art will recognize that there are a wide variety of reaction conditions that can be employed to amidate compounds of formula G, therefore, a wide variety of conditions are envisioned; see generally, March (2001); Larock (1999); Benz, G. “Synthesis of Amides and Related Compounds.” in Comprehensive Organic Synthesis, Trost, B. M., Editor, Pergamon Press: New York, N.Y., Vol. 6; and Bailey, P. D. et al. “Amides” in Comprehensive Organic Functional Group Transformation, Katritzky, et. al. Editors, Pergamon: New York, N.Y., Vol. 5.

In certain embodiments, the amidation is conducted by first activating the carboxylic acid to facilitate acylation (e.g., by reaction with SOCl₂ or similar reagents), and subsequently treating the activated species with a source of ammonia [e.g., ammonia gas or solution in tetrahydrofuran toluene, heptane, tert-butyl methyl ether, diethyl ether, ethyl acetate, isopropyl acetate, dichloromethane, chloroform, dichloroethan, or water (e.g., NH₄OH)]. In other embodiments, this reaction is conducted by first activating the carboxylic acid to facilitate acylation by reaction with SOCl₂ and subsequently treating the activated species with NH₄OH.

In still other embodiments, the reaction is run in toluene, benzene, ethyl acetate, dichloromethane, chloroform, dichloroethane, combinations thereof. In some embodiments, the cyclization is run in the absence of solvent.

In other embodiments, the reaction is run at a temperature between about −10° C. and about 150° C. In still other embodiments, the reaction is run at a temperature between about 50° C. and about 100° C.

In yet other embodiments, the reaction is conducted in a manner substantially similar to that described in Zhang, M. et al. Tetrahedron Lett. 2004, 45, 5229 or Devant, R. International patent application publication number WO05037817 (2005).

According to another embodiment, the present invention provides a method for preparing a compound of formula E: 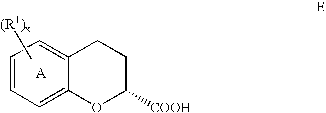
wherein:

x is 0-3;

each R¹ is independently —R, —CN, halogen or —OR; and

each R is independently hydrogen, C₁₋₆ aliphatic or C₁₋₆ haloaliphatic;

comprising the steps of:

(a) providing a compound of formula F: 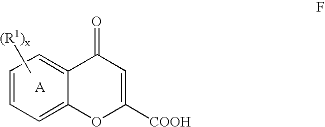
   wherein:

x is 0-3;

each R¹ is independently —R, —CN, halogen or —OR; and

each R is independently hydrogen, C₁₋₆ aliphatic or C₁₋₆ haloaliphatic;

and

(b) hydrogenating the compound of formula F to afford a compound of formula E.

For compounds of formula F, each of x and R¹ are as defined above in embodiments and subembodiments for compounds of formula II and II•HX.

In this step, a compound of formula F is reduced to afford a compound of formula E. In one aspect of the present invention, this step is carried out by (a) first subjecting the compound of formula F to hydrogenation conditions, (b) forming diastereomeric salts by combining the racemic mixture of the hydrogenation product with an enantioenriched chiral amine, (c) selectively crystallizing one of the diastereomeric salts to afford a diastereomerically enriched mixture of salts, and (d) recovering the acid in enantioenriched form from the diastereomerically enriched salt, as depicted in Scheme III, above. In certain embodiments, the hydrogenation in (a) is conducted in the presence of a palladium catalyst. In other embodiments, the palladium catalyst is palladium on carbon. In still other embodiments, the hydrogenation is run in methanol, ethanol, or acetic acid. According to one aspect of the present invention, the hydrogenation is run in methanol. In yet other embodiments, the hydrogenation is conducted in the presence of sulfuric acid, acetic acid, or both. In some embodiments, the hydrogenation is conducted in the presence of sulfuric acid. In still other embodiments, the hydrogenation is conducted in a manner substantially similar to that described in Witiak, D. T. et al. J. Med. Chem. 1975, 18, 934. In another aspect of the present invention, the enantioenriched chiral amine is (R)-1-phenyl-propylamine. In certain embodiments, the crystallization in step (c) is conducted in acetonitrile, methanol, ethanol, isopropanol, ethyl acetate, isopropyl acetate, diethyl ether, tert-butyl methyl ether, benzene, toluene, dichloromethane or the like. In certain embodiments, the free acid is liberated in step (d) by treating the salt with hydrochloric acid or sulfuric acid. In other embodiments, step (d) is conducted in toluene, water, or mixtures thereof. In other embodiments, the resolution step is conducted in a manner substantially similar to that described in Wigerinck, P. T. B. P. et al., International patent application number WO 9929687 A1 (1999); Van Lommen, G. R. E. et al., European patent application publication number EP 145067 A2 (1985); or Schaff, T. K. et al. J. Med. Chem. 1983, 26, 328.

In another aspect of the present invention, this step is carried out by (a) first subjecting the compound of formula F to hydrogenation conditions, (b) resolving the racemic reduced product by enzymatic means. In certain embodiments, the enzymatic resolution is carried out in a manner substantially similar to that described in Schutt, H., German patent application publication number DE 4430089 A1 (1996); Urban, F. J., European patent application publication number EP 0448254 A2 (1991); and Rossi, R. F., Jr., international patent application publication number WO 9640975 A1 (1996).

In yet another aspect of the present invention, this step is carried out by (a) hydrogenating a compound of formula F in an asymmetric fashion to afford an intermediate ketone-containing compound in enantiomerically enriched form, and (b) hydrogenating said intermediate to reduce the keto moiety and afford a compound of formula E in enantiomerically enriched form, as shown in Scheme IV, above. In certain embodiments, the asymmetric hydrogenation in step (a) is catalyzed by a suitable chiral catalyst. In certain embodiments, the chiral catalyst is a complex comprising a transition metal species and a suitable chiral ligand. In certain embodiments, the transition metal species is a late transition metal species (e.g., a Ru, Rh, Pd, Ir, or Pt species). In other embodiments the transition metal species is a rhodium or ruthenium species. In certain embodiments, the chiral ligand contains a phosphorus moiety that is capable of binding a transition metal species (e.g., a phosphine or phosphite moiety). In other embodiments the chiral ligand contains an olefinic moiety that is capable of binding a transition metal species. In yet other embodiments, the chiral ligand contains a carbene moiety that is capable of binding to a transition metal species. Suitable chiral ligands for asymmetric hydrogenation are well known in the art; see, e.g., Eliel (1994), Noyori (1994), Burgess (2005) Tang (2003). Additional exemplary chiral ligands include, but are not limited to, JosiPhos-type, MandyPhos™-type, WalPhos-type, TaniaPhos™-type, RoPhos-type, DIPAMP-type, Butiphane-type, BPE-type, QUINAP-type, BINAP-type, NorPhos-type, MonoPhos™-type, TunePhos-type, MalPhos-type, DuPhos-type, PHOX-type, KetalPhos-type, f-KetalPhos-type, TangPhos-type, BIPHEP-type, ferrotane-type, Binaphane-type, f-Binaphane-type, Binapine-type, FAP-type, MOP-type, DIOP-type, ChiraPhos-type, BPPM-type, and BICP-type. In certain embodiments the asymmetric hydrogenation is catalyzed by a chiral transition metal-containing species. In certain embodiments, the hydrogenation in step (b) is is conducted in the presence of a palladium catalyst. In other embodiments, the palladium catalyst is palladium on carbon. In still other embodiments, the hydrogenation is run in methanol. In yet other embodiments, the hydrogenation is conducted in the presence of sulfuric acid and acetic acid.

In certain embodiments, the chiral ligand employed in the asymmetric hydrogenation is selected from those depicted in Table I, above. In other embodiments, the chiral ligand is WalPhos W008-1.

According to another embodiment, the present invention provides a method for preparing a compound of formula E: 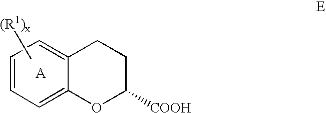
wherein:

x is 0-3;

each R¹ is independently —R, —CN, halogen or —OR; and

each R is independently hydrogen, C₁₋₆ aliphatic or C₁₋₆ haloaliphatic;

comprising the steps of:

(a) providing a compound of formula E-1: 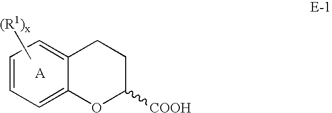
   wherein:

x is 0-3;

each R¹ is independently —R, —CN, halogen or —OR; and

each R is independently hydrogen, C₁₋₆ aliphatic or C₁₋₆ haloaliphatic;
(b) treating the compound of formula E-1 with a non-racemic chiral amine to afford a mixture of diastereomeric salts;
(c) selectively crystallizing one of the diastereomeric salts to afford a diastereomerically enriched mixture of salts; and
(d) recovering the compound of formula E in enantioenriched form from the diastereomerically enriched salt thereof.

For compounds of formula E and E-1, each of x and R¹ are as defined above in embodiments and subembodiments for compounds of formula II and II•HX.

In a certain embodiment, the compound E-1 is 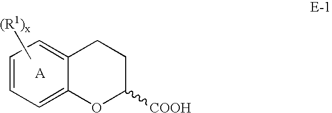
and the chiral non-racemic amine is (R)-1-phenylpropylamine. Amount of (R)-1-phenylpropylamine can be from 0.3 to 2 molar equivalents to the amount of the racemic acid, preferably, the amount is from 0.5 to 0.7 equivalents. In certain embodiments, the crystallization in step (c) is conducted in acetonitrile, methanol, ethanol, isopropanol, ethyl acetate, isopropyl acetate, diethyl ether, tert-butyl methyl ether, benzene, toluene, dichloromethane or the like. In certain embodiments, the free acid is liberated in step (d) by treating the salt with hydrochloric acid or sulfuric acid. In other embodiments, step (d) is conducted in toluene, water, or mixtures thereof. In other embodiments, the resolution step is conducted in a manner substantially similar to that described in Wigerinck, P. T. B. P. et al., International patent application number WO 9929687 A1 (1999); Van Lommen, G. R. E. et al., European patent application publication number EP 145067 A2 (1985); or Schaff, T. K. et al. J Med. Chem. 1983, 26, 328.

According to another embodiment, the present invention provides a method for preparing a compound of formula F: 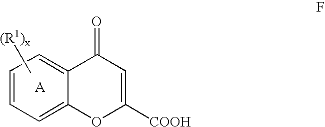
wherein:

x is 0-3;

each R¹ is independently —R, —CN, halogen or —OR; and

each R is independently hydrogen, C₁₋₆ aliphatic or C₁₋₆ haloaliphatic; comprising the steps of:
(a) providing a compound of formula G: 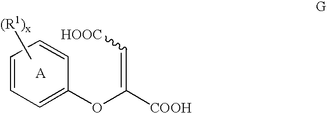
   wherein:

x is 0-3;

each R¹ is independently —R, —CN, halogen or —OR; and

each R is independently hydrogen, C₁₋₆ aliphatic or C₁₋₆ haloaliphatic;

and

(b) cyclizing the compound of formula G to afford a compound of formula F.

For compounds of formula G, each of x and R¹ are as defined above in embodiments and subembodiments for compounds of formula II and II•HX.

In this cyclization step, a compound of formula G is cyclized to afford a compound of formula F. One of ordinary skill in the art will recognize that there are a wide variety of reaction conditions that can be employed to cyclize compounds of formula G, therefore, a wide variety of conditions are envisioned; see generally, Smith and March, (2001) and Larock (1999). In certain embodiments, the cyclization is promoted by treating a compound of formula G with a suitable Brønsted acid. Exemplary acids include hydrochloric, sulfuric, phosphoric, polyphosphoric, methanesulfonic, Eaton's reagent (P₂O₅/MeSO₃H), chlorosulfonic, camphorsulfonic, and p-toluenesulfonic. In other embodiments, additional reagents are employed, including, for example, phosphorus pentoxide, phosphorus trichloride, phosphorus pentachloride, acetyl chloride, or acetic anhydride. One of ordinary skill in the art will recognize that some of the conditions described will promote formation of an intermediate acylchloride prior to undergoing cyclization. In yet another embodiment, the reaction is conducted with acetyl chloride or water as solvent. In still other embodiments, the cyclization is conducted in a manner substantially similar to that described in Ruhemann (1900), Gudi (1969), Cairns (1972), Stoermer (1995), or Fitzmaurice, C. et al. British Patent No. 1262078, (filed 24 May, 1968).

According to another embodiment, the present invention provides a method for preparing a compound of formula G: 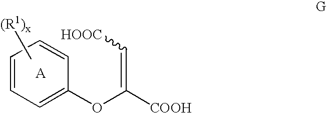
wherein:

x is 0-3;

each R¹ is independently —R, —CN, halogen or —OR; and

each R is independently hydrogen, C₁₋₆ aliphatic or C₁₋₆ haloaliphatic; comprising the steps of:
(a) providing a compound of formula H: 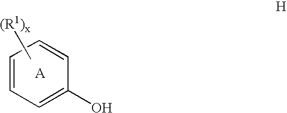
   wherein:

x is 0-3;

each R¹ is independently —R, —CN, halogen or —OR; and

each R is independently hydrogen, C₁₋₆ aliphatic or C₁₋₆ haloaliphatic;
(b) allowing said compound of formula H to react with a compound of formula J: 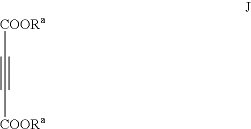
   wherein:

x is 0-3;

each R¹ is independently —R, —CN, halogen or —OR;

each R is independently hydrogen, C₁₋₆ aliphatic or C₁₋₆ haloaliphatic; and

each R^{a} is hydrogen, C₁₋₆ aliphatic, phenyl, benzyl, or tri(C₁₋₆ aliphatic)silyl,

and

(c) removing the R^{a} groups from the product of the reaction between the compound of formula H and the compound of formula J to afford a compound of formula G.

For compounds of formula H, each of x and R¹ are as defined above in embodiments and subembodiments for compounds of formula II and II•HX. As defined above, for compounds of formula J, each R^{a} group is independently hydrogen, C₁₋₆ aliphatic, phenyl, benzyl, or tri(C₁₋₆ aliphatic)silyl. In certain embodiments, each R^{a} is independently selected from ethyl, methyl, hydrogen, tert-butyl, or trimethylsilyl. In other embodiments, each R^{a} is ethyl.

In this step, a compound of formula H is allowed to react via conjugate addition with a compound of formula J, following which the R^{a} groups are removed to afford the product of formula G, as depicted in Scheme II, above. One of ordinary skill in the art will appreciate that a wide variety of reaction conditions may be employed to promote this transformation, therefore a wide variety of reaction conditions are envisioned; see generally, March (2001) and Larock (1999). For example, the conjugate addition step may be run in the presence or absence of a base, and with or without heating. In certain embodiments, the conjugate addition is run in the presence of potassium carbonate, potassium hydroxide, sodium hydroxide, tetrabutylammonium hydroxide, benzyltrimethylammonium hydroxide, triethylbenzylammonium hydroxide, 1,1,3,3-tetramethylguanidine, 1,8-diazabicyclo[5.4.0]undec-7-ene, N-methylmorpholine, diisopropylethylamine, tetramethylethylenediamine, pyridine, or triethylamine.

In certain embodiments, the reaction is carried out in a suitable medium. In certain embodiments the present transformation is run in excess of the phenol reagent (corresponding to formula H), diphenyl ether, dioxane, anisole, acetone, tetrahydrofuran, ethyl acetate, isopropyl acetate, dimethylformamide, ethylene glycol, toluene, water, diisopropylethylamine, triethylamine, pyridine, N-methylmorpholine, acetonitrile, N-methylpyrrolidine, or mixtures thereof.

In other embodiments the reaction is conducted at temperatures between about 25° C. and about 110° C. In yet other embodiments, the reaction is conducted at about 25° C.

In other embodiments, the conjugate addition is carried out in a manner substantially similar to that described in Ruhemann (1900), Gudi (1969), Cairns (1972), Stoermer (1995) or Fitzmaurice, C. et al. British Patent No. 1262078, (filed 24 May, 1968).

One of ordinary skill in the art will recognize that there are a wide variety of reaction conditions that can be employed to remove the R^{a} groups to afford compounds of formula G, therefore, a wide variety of conditions are envisioned; see generally, Smith and March, (2001) and Larock (1999). For example, the removal the R^{a} groups can be promoted by reaction with base (e.g., sodium hydroxide, tetrabutylammonium hydroxide, or the like) or acid (e.g., hydrochloric acid, sulfuric acid, acetic acid, camphorsulfonic acid, p-toluenesulfonic acid, or the like), with sources of fluoride (e.g., tetrabutylammonium fluoride, potassium fluoride, pyridinium fluoride, triethylammonium fluoride, tetrabutylammonium triphenyldifluorosilicate, or the like), and optionally with heating of the reaction mixture. In certain embodiments, the removal of the R^{a} groups is promoted by reaction with sodium hydroxide. In other embodiments, this reaction is conducted at a temperature of between about 40° C. and about 100° C.

According to another aspect, the present invention provides a method for preparing a compound of formula II•HX: 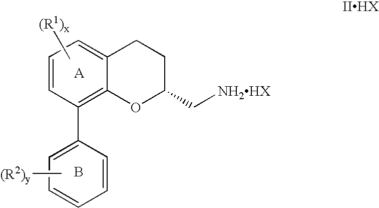
wherein:
x is 0-3;
y is 0-5;
each R¹ is independently —R, —CN, halogen or —OR;
each R is independently hydrogen, C₁₋₆ aliphatic or C₁₋₆ haloaliphatic;
each R² is independently -Ph, halogen, —CN, —R or —OR; and
X is the anion of a suitable acid,
   comprising the steps of:
(a) providing a compound of formula H: 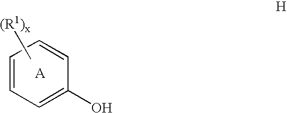
   wherein:
   x is 0-3;
   each R¹ is independently —R, —CN, halogen or —OR; and
   each R is independently hydrogen, C₁₋₆ aliphatic or C₁₋₆ haloaliphatic;
(b) allowing said compound of formula H to react with a compound of formula J: 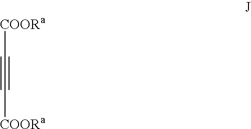
   wherein:
   x is 0-3;
   each R¹ is independently —R, —CN, halogen or —OR;
   each R is independently hydrogen, C₁₋₆ aliphatic or C₁₋₆ haloaliphatic; and
   each R^{a} is hydrogen, C₁₋₆ aliphatic, phenyl, benzyl, or tri(C₁₋₆ aliphatic)silyl,
(c) removing the R^{a} groups from the product of the reaction between the compound of formula H and the compound of formula J to afford a compound of formula G: 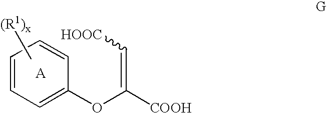
   wherein:
   x is 0-3;
   each R¹ is independently —R, —CN, halogen or —OR; and
   each R is independently hydrogen, C₁₋₆ aliphatic or C₁₋₆ haloaliphatic;
(d) cyclizing the compound of formula G to afford a compound of formula F: 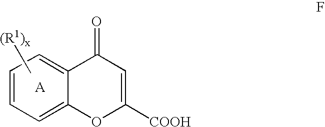
   wherein:
   x is 0-3;
   each R¹ is independently —R, —CN, halogen or —OR; and
   each R is independently hydrogen, C₁₋₆ aliphatic or C₁₋₆ haloaliphatic;
(e) hydrogenating the compound of formula F to afford a compound of formula E: 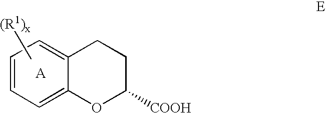
   wherein:
   x is 0-3;
   each R¹ is independently —R, —CN, halogen or —OR; and
   each R is independently hydrogen, C₁₋₆ aliphatic or C₁₋₆ haloaliphatic;
(f) converting the carboxylic acid moiety in the compound of formula E into an amide moiety to form a compound of formula D: 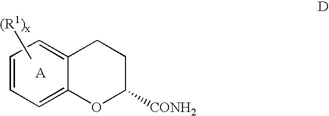
   wherein:
   x is 0-3;
   each R¹ is independently —R, —CN, halogen or —OR; and
   each R is independently hydrogen, C₁₋₆ aliphatic or C₁₋₆ haloaliphatic;
(g) reducing the amide moiety in the compound of formula D to the amine;
(h) optionally protecting the amine moiety resulting from the reduction of the amide moiety in the compound of formula D with a suitable amine protecting group to afford a compound of formula C: 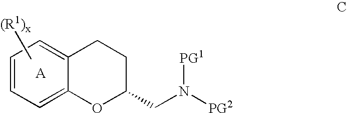
   wherein:
   x is 0-3;
   each R¹ is independently —R, —CN, halogen or —OR;
   each R is independently hydrogen, C₁₋₆ aliphatic or C₁₋₆ haloaliphatic; and
   PG¹ and PG² are each independently hydrogen or a suitable amino protecting group,
(i) introducing a CG¹ group at the open ortho position relative to the sp2-hybridized carbon bearing the chromane oxygen in formula C to afford a compound of formula A: 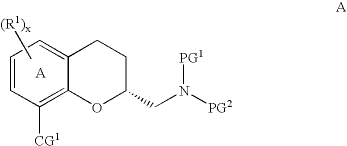
   wherein:
   x is 0-3;
   each R¹ is independently —R, —CN, halogen or —OR;
   each R is independently hydrogen, C₁₋₆ aliphatic or C₁₋₆ haloaliphatic;
   PG¹ and PG² are each independently hydrogen or a suitable amino protecting group; and
   CG¹ is a coupling group that facilitates transition metal-mediated Cₛₚ₂-Cₛₚ₂ coupling between the attached Cₛₚ₂ carbon and a Cₛₚ₂ carbon bearing a CG² coupling group,
(j) coupling said compound of formula A with a compound of formula B: 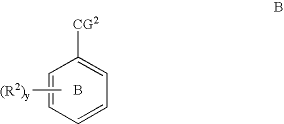
   wherein:
   y is 0-5;
   each R is independently hydrogen, C₁₋₆ aliphatic or C₁₋₆ haloaliphatic; and
   each R² is independently -Ph, halogen, —CN, —R or —OR,
   CG² is a coupling group that facilitates transition metal-mediated Cₛₚ₂-Cₛₚ₂ coupling between the attached Cₛₚ₂ carbon and a Cₛₚ₂ carbon bearing a CG¹ coupling group;
      in the presence of a suitable transition metal,
(k) deprotecting the protected amine moiety of the coupling product to form a compound of formula II: 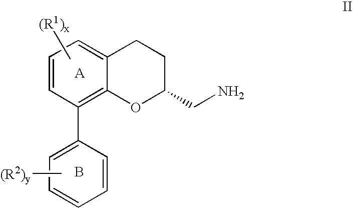
   wherein:
   x is 0-3;
   y is 0-5;
   each R¹ is independently —R, —CN, halogen or —OR;
   each R is independently hydrogen, C₁₋₆ aliphatic or C₁₋₆ haloaliphatic; and
   each R² is independently -Ph, halogen, —CN, —R or —OR,
      and
(l) reacting said compound of formula II with suitable acid of formula HX to form a compound of formula II•X.

According to another aspect, the present invention provides a method for preparing a compound of formula II•HX: 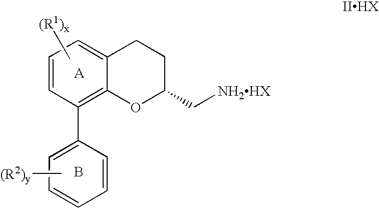
wherein:
x is 0-3;
y is 0-5;
each R¹ is independently —R, —CN, halogen or —OR;
each R is independently hydrogen, C₁₋₆ aliphatic or C₁₋₆ haloaliphatic;
each R² is independently -Ph, halogen, —CN, —R or —OR; and
X is the conjugate base of a suitable acid,
   comprising the steps of:
(a) providing a compound of formula C: 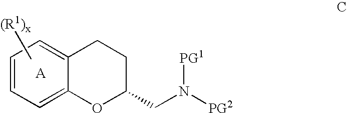
   wherein:
   x is 0-3;
   each R¹ is independently —R, —CN, halogen or —OR;
   each R is independently hydrogen, C₁₋₆ aliphatic or C₁₋₆ haloaliphatic; and
   PG¹ and PG² are each independently hydrogen or a suitable amino protecting group,
(b) introducing a CG¹ group at the open ortho position relative to the sp2-hybridized carbon bearing the chromane oxygen in formula C to afford a compound of formula A: 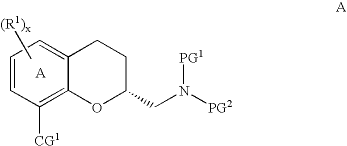
   wherein:
   x is 0-3;
   each R¹ is independently —R, —CN, halogen or —OR;
   each R is independently hydrogen, C₁₋₆ aliphatic or C₁₋₆ haloaliphatic;
   PG¹ and PG² are each independently hydrogen or a suitable amino protecting group; and
   CG¹ is a coupling group that facilitates transition metal-mediated Cₛₚ₂-Cₛₚ₂ coupling between the attached Cₛₚ₂ carbon and a Cₛₚ₂ carbon bearing a CG² coupling group,
(c) coupling said compound of formula A with a compound of formula B: 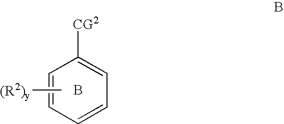
   wherein:
   y is 0-5;
   each R is independently hydrogen, C₁₋₆ aliphatic or C₁₋₆ haloaliphatic;
   each R² is independently -Ph, halogen, —CN, —R or —OR; and
   CG² is a coupling group that facilitates transition metal-mediated Cₛₚ₂-Cₛₚ₂ coupling between the attached Cₛₚ₂ carbon and a Cₛₚ₂ carbon bearing a CG¹ coupling group;
      in the presence of a suitable transition metal,
(d) deprotecting the optionally protected amine moiety of the coupling product to form a compound of formula II: 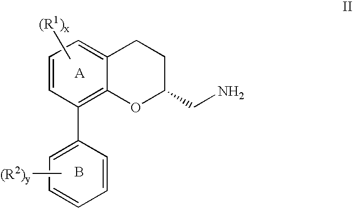
   wherein:
   x is 0-3;
   y is 0-5;
   each R¹ is independently —R, —CN, halogen or —OR;
   each R is independently hydrogen, C₁₋₆ aliphatic or C₁₋₆ haloaliphatic; and
   each R² is independently -Ph, halogen, —CN, —R or —OR,
      and
(e) reacting said compound of formula II with suitable acid of formula HX to form a compound of formula II•X.

In certain embodiments, the present invention provides a method for preparing a compound of formula O: 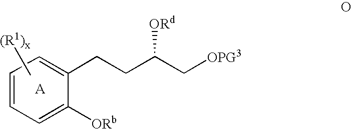
wherein:
x is 0-3;
each R¹ is independently —R, —CN, halogen or —OR;
each R is independently hydrogen, C₁₋₆ aliphatic or C₁₋₆ haloaliphatic;
R^{b} is hydrogen or a hydroxyl protecting group;
R^{d} is hydrogen or a suitable hydroxyl protecting group; and
PG³ is a suitable hydroxyl protecting group,
   comprising the steps of:
(a) providing a compound of formula Q: 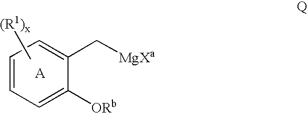
   wherein:
   x is 0-3;
   X^{a} is halogen;
   each R¹ is independently —R, —CN, halogen or —OR;
   each R is independently hydrogen, C₁₋₆ aliphatic or C₁₋₆ haloaliphatic; and
   R^{b} is a suitable hydroxyl protecting group,
      and
(b) reacting the compound of formula Q with a non-racemic compound of formula P: 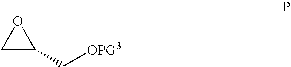
   wherein PG³ is a hydroxyl protecting group, to form the compound of formula O.

For compounds of formulae Q, O, and P, each of x, R¹, PG³, R^{b}, R^{d}, and X^{a} are as defined above and described in embodiments and subembodiments above and herein.

In other embodiments, the present invention provides a method for preparing a compound of formula N: 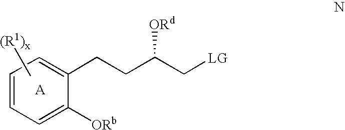
wherein:
x is 0-3;
each R¹ is independently —R, —CN, halogen or —OR;
each R is independently hydrogen, C₁₋₆ aliphatic or C₁₋₆ haloaliphatic;
R^{b} is hydrogen or a suitable hydroxyl protecting group;
R^{d} is hydrogen or a suitable hydroxyl protecting group; and
LG is a suitable leaving group,
   comprising the steps of:
(a) providing a compound of formula O: 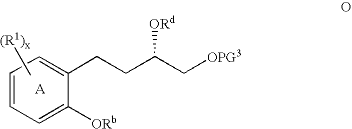
   wherein:
   x is 0-3;
   y is 0-5;
   each R¹ is independently —R, —CN, halogen or —OR;
   each R is independently hydrogen, C₁₋₆ aliphatic or C₁₋₆ haloaliphatic;
   each R² is independently -Ph, halogen, —CN, —R or —OR;
   R^{b} is hydrogen or a suitable hydroxyl protecting group;
   R^{d} is hydrogen or a suitable hydroxyl protecting group; and
   PG³ is a hydroxyl protecting group,
(b) removing PG³ and converting the free hydroxyl moiety into a suitable leaving group to afford the compound of formula N.

For compounds of formulae O, and N, each of x, R¹, PG³, R^{b}, R^{d}, and LG are as defined above and described in embodiments and subembodiments above and herein.

In certain embodiments, the present invention provides a method for preparing a compound of formula L: 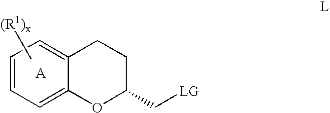
wherein:
x is 0-3;
each R¹ is independently —R, —CN, halogen or —OR;
each R is independently hydrogen, C₁₋₆ aliphatic or C₁₋₆ haloaliphatic; and
LG is a suitable leaving group,
   comprising the steps of:
(a) providing a compound of formula N: 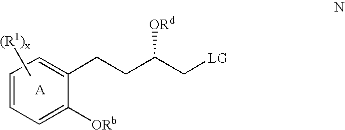
   wherein:
   x is 0-3;

each R¹ is independently —R, —CN, halogen or —OR;
each R is independently hydrogen, C₁₋₆ aliphatic or C₁₋₆ haloaliphatic;
R^{b} is hydrogen or a suitable hydroxyl protecting group;
R^{d} is hydrogen or a suitable hydroxyl protecting group; and
LG is a suitable leaving group,
(b) removing protecting group R^{b} and, if present, R^{d}, to form a diol of formula M 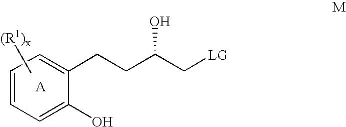
   wherein:
   x is 0-3;
   each R¹ is independently —R, —CN, halogen or —OR;
   each R is independently hydrogen, C₁₋₆ aliphatic or C₁₋₆ haloaliphatic; and
   LG is a suitable leaving group,
      and
(c) cycling the compound of formula M to form the compound of formula L.

For compounds of formulae Q, O, and P, each of x, R¹, LG, R^{b}, and R^{d} are as defined above and described in embodiments and subembodiments above and herein.

According to another embodiment, the present invention provides a method for preparing a compound of formula C: 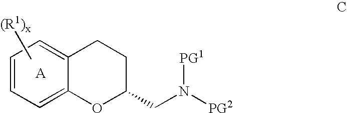
wherein:
x is 0-3;
each R¹ is independently —R, —CN, halogen or —OR;
each R is independently hydrogen, C₁₋₆ aliphatic or C₁₋₆ haloaliphatic; and
PG¹ and PG² are each hydrogen or a suitable protecting group, comprising the steps of:
(a) providing a compound of formula L: 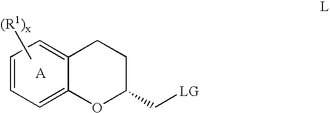
   wherein:
   x is 0-3;
   each R¹ is independently —R, —CN, halogen or —OR;
   each R is independently hydrogen, C₁₋₆ aliphatic or C₁₋₆ haloaliphatic; and
   LG is a suitable leaving group,
      and
(b) treating the compound of formula L with a suitable amine to afford the compound of formula C.
For the above method, according to another embodiment, the present invention provides a method for preparing a compound of formula C further comprising the step of:
(c) protecting said suitable amine with a suitable amino protecting group to afford the compound of formula C;
   wherein at least one of PG¹ or PG² is a suitable protecting group.

For compounds of formulae C and L, each of x, R¹, PG¹, PG², and LG are as defined above and described in embodiments and subembodiments above and herein.

Yet another aspect of the present invention provides a compound of formula A: 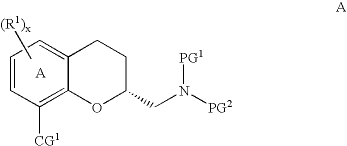
wherein:
x is 0-3;
each R¹ is independently —R, —CN, halogen or —OR;
each R is independently hydrogen, C₁₋₆ aliphatic or C₁₋₆ haloaliphatic; and
PG¹ and PG² are each independently hydrogen or a suitable protecting group.

For compounds of formula A, each of x, R¹, PG¹, PG², and CG¹ are as defined in embodiments and subembodiments herein. According to one aspect of the present invention, the compound of formula A is

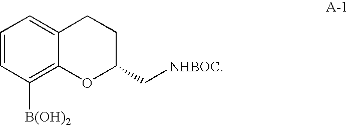

Yet another aspect of the present invention provides a compound of formula II: 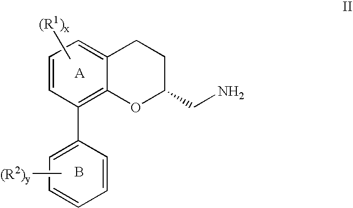
wherein:
x is 0-3;
y is 0-5;
each R¹ is independently —R, —CN, halogen or —OR;
each R is independently hydrogen, C₁₋₆ aliphatic or C₁₋₆ haloaliphatic; and
each R² is independently -Ph, halogen, —CN, —R or —OR.

For compounds of formula II, each of x, y, R¹, and R² are as defined in embodiments and subembodiments herein. According to one aspect of the present invention, the compound of formula II is

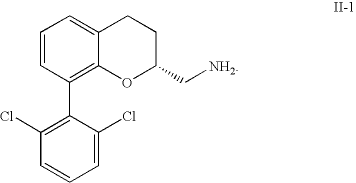

Yet another aspect of the present invention provides a compound of formula II•HX: 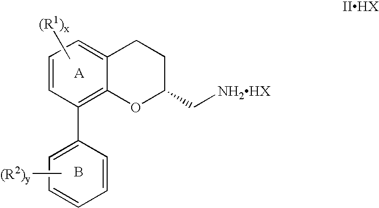
wherein:
x is 0-3;
y is 0-5;
each R¹ is independently —R, —CN, halogen or —OR;
each R is independently hydrogen, C₁₋₆ aliphatic or C₁₋₆ haloaliphatic;
each R² is independently -Ph, halogen, —CN, —R or —OR; and
X is the anion of a suitable acid.

For compounds of formula II•HX, each of x, y, R¹, R², and X are as defined in embodiments and subembodiments herein. According to one aspect of the present invention, the compound of formula II•HX is

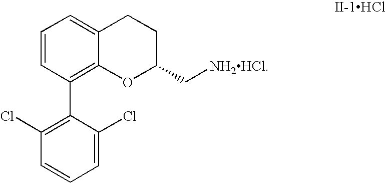

### EXAMPLES

### Example 1

### Preparation of (R)-2-(Aminomethyl)-8-(2,6-dichlorophenyl)chromane hydrochloride:

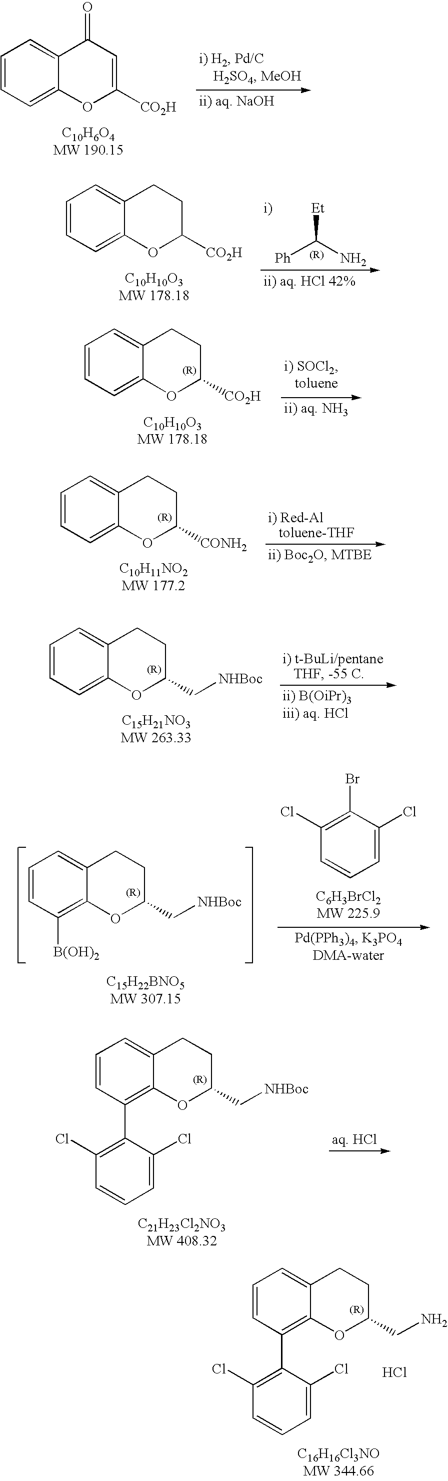

Unless otherwise indicated below, NMR spectra of the intermediates were recorded on a Bruker Avance DPX300 or DRX400 NMR spectrometers. Spectra were referenced by an internal standard.

HPLC analysis of the intermediates and reaction monitoring was carried out on an Agilent 1090 liquid chromatograph equipped a Phenomenex 4.6×50 mm Prodigy ODS3 column. Standard method: 90:10 to 10:90 9 min gradient of water-acetonitrile containing 0.02% TFA, flow rate 1 ml/min.

HPLC analysis of the final compound was done on an Agilent 1100 series chromatograph equipped with a Prodigy ODS3, 0.46×15 cm column. Standard method: 90:10 to 10:90 20 min gradient of water-acetonitrile containing 0.02% TFA, flow rate 1 ml/min. Enantiomeric purities were determined by HPLC on an Agilent 1100 series chromatograph. Conditions for individual compounds are described below together with the experimental data. LCMS data were obtained on an Agilent 1100 LC system with an Agilent 1100 LC/MS detector equipped with a Phenomenex Capcell Pak 5u C8 4.6×50 mm column. Standard method: 90:10 to 10:90 8 min gradient of water-acetonitrile containing 0.02% HCO₂H, flow rate 1 ml/min.

High resolution mass spectra (HRMS) were recorded on a Bruker Apex II 9.4T FTMS spectrometer using ES ionization mode.

Optical rotation was measured on Jasco P-1020 polarimeter.

### rac-Chroman-2-carboxylic acid

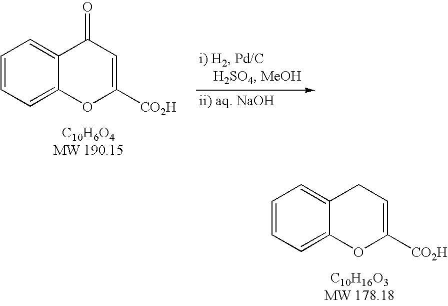

A 2.5 L Parr shaker vessel was charged with chromone-2-carboxylic acid (150.0 g, 0.789 mol), a slurry of palladium on carbon (10% wet, Aldrich, 8.36 g, 3.9 mmol Pd, 0.5 mol %) in methanol (20 mL). Concentrated sulfuric acid (46 mL, 0.83 mol) was dissolved in 1.00 L of methanol (Very exothermic!) and the solution was added to the mixture in the Parr vessel.

The mixture was hydrogenated on a Parr shaker at 50 psi hydrogen pressure and room temperature. Hydrogen reservoir was refilled to 50 psi whenever pressure dropped below 40 psi. The reaction was monitored by HPLC. Hydrogenation was continued until the starting material became undetectable (<0.1% at 215 nm), usually overnight.

The catalyst was removed by filtering the reaction mixture through a pad of Celite. The filtrate containing methyl chromane-2-carboxylate was transferred into a 4-liter Erlenmeyer flask equipped with a mechanical stirrer. Water (0.50 L) and 10 M aqueous solution of NaOH (300 mL, 3.0 mol) were added (mild exotherm, 42° C., precipitate of sodium sulfate separated) and the mixture was stirred for 10 min at room temperature at which point HPLC analysis showed complete hydrolysis of the ester (<0.1% at 215 nm). The inorganic precipitate was filtered off and methanol was removed from the solution on rotary evaporator. The residue after evaporation was mixed with MTBE (250 mL) and the mixture was acidified with 6 M aq. HCl until pH of the aqueous layer fell below 3 (ca. 300 mL). The aqueous layer was separated and extracted with MTBE (2×300 mL). The extracts, combined with the organic layer, were washed with brine (200 mL) and dried with MgSO4. The drying agent was filtered off and the filtrate was evaporated to a thick oil (180 g). This residue was triturated with heptane (50 mL) which caused rapid crystallization of the acid. The mixture was chilled in ice, filtered and the solid on the filter was washed with cold heptane and dried on the filter in a stream of air. Yield of the chromane-2-carboxylic acid 117.6 g (85%) as colorless crystals. Purity 99% (HPLC, 215 nm). A second crop of crystals was obtained by evaporation of the filtrate to 30 mL and filtration of the separated crystals: 10.7 g (8%). Purity 94% (HPLC, 215 nm, 2 major impurities 2.8 and 3.4%).

### rac-Chroman-2-carboxamide

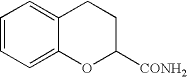

Chromone-2-carboxylic acid (25.0 g, 0.132 mol) was suspended in methanol (150 mL) in a 2.5 L Parr shaker vessel. To the mixture were added palladium on carbon (10% wet, Aldrich, 1.50 g, 0.71 mmol Pd, 0.5 mol %) as a suspension in methanol (20 mL) and a solution of conc. sulfuric acid (7.5 mL, 0.135 mol) in methanol (80 mL).

The mixture was hydrogenated on a Parr shaker at room temperature at 50 psi. The reaction was monitored by HPLC and was complete after running overnight (hydrogen was refilled to 48 psi when the pressure dropped below 40 psi).

After the hydrogenation was complete, conc. aqueous ammonia solution (18 mL, 0.27 mol) was added to the reaction mixture containing methyl chromane-2-carboxylate. The slurry of ammonium sulfate and the catalyst was filtered and the filtrate was transferred into a high-pressure bottle equipped with a magnetic stirrer, a pressure gauge mounted into the screw cap. More conc. aq. ammonia was added to the vessel (90 mL, 1.35 mol), the vessel was capped tightly and heated in a 45° C. oil bath for 19 hr (the gauge registered almost no pressure, <3 psi).

The solution was allowed to cool to room temperature before the vessel was opened. The solvent was removed on a rotary evaporator. The residue was diluted with 50 mL of water which caused precipitation of the product as a white solid. The slurry was chilled in ice and then filtered. The solid on the filter was washed with 100 mL of water and dried in air. Yield 19.8 g (85%). Purity >99% (HPLC, 215 nm). M.p. 128-130° C.

¹H NMR (300 MHz, CDCl3), δ: 7.06-7.17 (m, 2H), 6.94-6.85 (m, 2H), 6.60 (broad s, 1H), 5.64 (broad s, 1H), 4.55 (dd, J=3.1, 9.3 Hz, 1H), 2.96-2.75 (m, 2H), 2.41 (m, 1H), 2.09 (m, 1H).

### (R)-(−)-Chromane-2-carboxylic acid (chemical resolution)

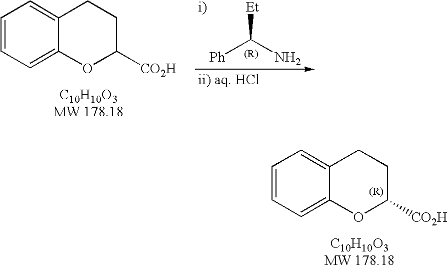

A 1-L Erlenmeyer flask equipped with a magnetic stirrer was charged with racemic chromane-2-carboxylic acid (60.9 g, 0.342 mol) and acetonitrile (300 mL). In a separate flask, (R)-(+)-1-phenylpropylamine (30.0 g, 0.222 mol, 0.65 eq.) was mixed with 40 mL of MTBE and this solution was added portionwise to the solution of the acid via pipette. After about half of the total amount of the amine solution was added, the reaction mixture was seeded with a few crystals of (R)-(+)-1-phenylpropylammonium (R)-(−)-chromane-2-carboxylate. When precipitation of the salt began, the remainder of the amine solution was added over about 5 min. The thick slurry of crystalline salt was diluted with 260 mL of MTBE (total volume of MTBE added was 300 mL) and the mixture was stirred gently for 6 hr at room temperature.

The salt was filtered, washed with MTBE and dried in air. Yield 45.2 g (acid content 25.7 g, 42%). Ratio of the acid enantiomers 98:2 (see note below for analytical determination).

The salt (45.13 g, 0.144 mol) was placed into a 1-L Erlenmeyer flask equipped with a magnetic stirrer and was suspended in MTBE (250 mL). Aqueous 1 M HCl solution (290 mL, 0.290 mol) was added and the mixture was stirred at room temperature until all solid dissolved and 2 clear phases formed (about 10 min). The layers were separated. The aq. layer was extracted with MTBE (2×100 mL). The extracts were combined with the organic layer and the solution was washed with 0.5 M aq. HCl (100 mL), then brine (2×100 mL). The solution was then dried with MgSO4, the drying agent was filtered and the filtrate was concentrated in vacuum to ca. 50 mL. Heptane (50 mL) was mixed with the residue and crystallization of the product was induced by seeding it a few crystals of the optically pure acid. The solvents were then removed in vacuum. Yield 25.58 g (42% from the racemic acid, 99% from the salt) as white crystals. M.p. 81-83° C.

¹H NMR (400 MHz, CDCl₃), δ: 9.23 (br s, 1H), 7.13 (t, J=7.8 Hz, 1H), 7.06 (d, J=7.6 Hz, 1H), 6.95-6.87 (m, 2H), 4.76 (dd, J=3.5, 8.1 Hz, 1H), 2.93-2.76 (m, 2H), 2.41-2.32 (m, 1H), 2.25-2.14 (m, 1H).

¹³C NMR (100 MHz, CDCl₃), δ: 175.43, 152.85, 129.53, 127.73, 121.29, 121.18, 116.85, 73.29, 24.49, 23.40.

HRMS (ESI), m/z: 177.05519 (M−H)⁻; calc'd for C₁₀H₉O₃: 177.05572.

Enantiomeric purity determination. HPLC method: Chiralcel ODH, 0.46×25 cm column, isocratic 75% heptane/TFA, 25% Ethanol, distomer 4.4 min, eutomer 6.7 min.

### (R)-Chroman-2-carboxamide

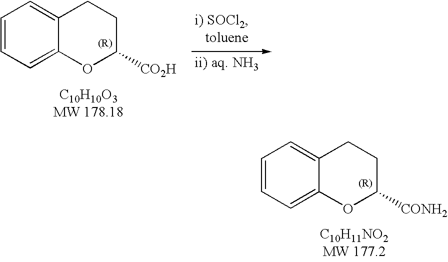

(R)-Chromane-2-carboxylic acid (25.45 g, 0.143 mol) was mixed with toluene (75 mL) in a 1-L round bottom flask equipped with a magnetic stirbar. Neat thionyl chloride (52 mL, 0.715 mol, 5.0 eq.) was added and the mixture was heated in an oil bath at 65° C. (bath temperature) for 18 hr. The volatile components of the mixture were removed in vacuum (40° C.) and chased by 70 mL of toluene. The yellow oily residue was dissolved in 70 mL of toluene.

Conc. aq. ammonia (89 mL, 1.43 mol) and THF (150 mL) was placed into a 1-L Erlenmeyer flask, equipped with a magnetic stirbar and temperature probe, and chilled to 0° C. in an acetone-dry ice bath (bath temp. −20° C.).

The solution of acid chloride in toluene was added to the ammonia solution portionwise (5-7 mL) via a pipette at a rate to keep the inside temperature below 5° C. (bath temperature was maintained at about −20° C.). When all of the acid chloride was added, the organic layer was separated (more THF may need to be added if some product precipitates out) and concentrated on rotary evaporator until most of THF was removed (solid product precipitated out). The residue after evaporation was dissolved in ethyl acetate (about 500 mL). The solution was washed with water (50 mL), brine (50 mL), and was dried with MgSO4. The drying agent was filtered off and the filtrate was evaporated to a thick paste. The residue was mixed with 50 mL of heptane and the mixture was again evaporated to a thick paste. The slurry was chilled in ice and was filtered. The solid on the filter was washed cold heptane and dried in air: 23.0 g (91%) as white crystals. Analytical purity >99% (HPLC), enantiomer ratio R:S 99:1 (see note below for description of enantiomeric purity determination). M.p. 140-141° C.

A second crop of crystals was obtained when the filtrate was concentrated in vacuum to 30 mL, diluted with 50 mL of heptane and evaporated again to 30 mL. The crystals were filtered, washed with heptane-MTBE 2:1 mixture and dried in air: 1.12 g (4%) as off-white crystals. Enantiomer ratio R:S 76:24.

¹H NMR (400 MHz, CDCl₃), δ: 7.13 (m, 1H), 7.08 (m, 1H), 6.60 (br s, 1H), 5.68 (br s, 1H), 4.55 (dd, J=3.0, 9.3 Hz, 1H), 2.94-2.76 (m, 2H), 2.45-2.37 (m, 1H), 2.13-2.02 (m, 1H).

¹³C NMR (100 MHz, CDCl₃), δ: 173.47, 152.88, 129.78, 127.57, 122.07, 121.32, 116.61, 75.33, 24.72, 24.01.

HRMS (ESI+), m/z: 178.08588 (M+H),⁺ calc'd for C₁₀H₁₂NO₂: 178.08626.

Enantiomeric purity determination. HPLC method: Chiralcel OJH, 0.46×25 cm column, isocratic 85% Heptane/DEA, 15% IPA, 1.0 mL/min; eutomer 9.7 min, distomer 11.2 mm.

### (R)-tert-butyl(2-chromanyl)methylcarbamate

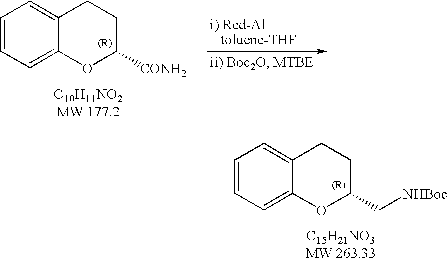

A 1-L round bottom flask equipped with a mechanical stirrer, a rubber septum, a 250-mL addition funnel, a temperature probe and a nitrogen line and placed into an acetone cold bath was purged with nitrogen and then charged with a solution of Red-Al in toluene (60 wt %, Aldrich, 118 mL, 0.39 mol, 3.0 eq.). A solution of (R)-chromane-2-carboxamide in 230 mL of dry THF was transferred into the addition funnel. The contents of the flask were chilled to −5° C. (bath temp. was maintained at −20° C. with dry ice) and the amide solution was added slowly to the reducing agent at a rate to maintain the reaction temperature below 0° C. (addition time 25 min). Resulting solution was allowed to warm to room temperature and was left overnight. (Note: The reaction flask should not be taken out of the bath as the reaction exotherm can warm the reaction mixture to over 40° C. which may cause racemization of the unreacted amide). In 18 hrs, HPLC analysis showed complete conversion of the starting material to the product.

A 2-L round bottom flask equipped with a mechanical stirrer, a temperature probe and a rubber septum was charged with water (0.60 L) and 10 M aq. solution of NaOH (150 mL, 1.5 mol). The flask was set in a −25° C. acetone-dry ice bath. When the NaOH solution cooled to below 0° C., the reaction mixture from the first flask was being added slowly into the NaOH solution via an 18-gauge cannula (Exotherm, hydrogen evolution!). The rate of transfer was regulated by nitrogen pressure in the first reaction flask and was adjusted to keep the quench temperature below 0° C., the bath temperature was maintained in the −20 to −25° C. range. When all of the reaction mixture was quenched, the cold bath was removed and the bi-phasic mixture was stirred until it reached room temperature. The layers were separated. The organic layer was concentrated in vacuum to remove most of THF. The residue was dissolved in 300 mL of MTBE. The solution was washed with 1 M aq. NaOH (50 mL), brine (2×50 mL) and was dried with Na₂SO₄. The drying agent was filtered and the filtrate was placed into a 300 mL Erlenmeyer flask equipped with a magnetic stirrer and a temperature probe.

A solution of Boc anhydride (28.3 g, 0.13 mol) in MTBE (total solution volume 110 mL) was added to the solution of amine via pipette. A thick white precipitate separated from the solution at the beginning of the addition but gradually dissolved after the addition was complete. Very mild exotherm was observe during the addition: reaction temperature went up by 5° C. to 24° C.

In 15 min after the addition was complete, HPLC showed complete conversion of the amine to its Boc derivative. The reaction mixture was treated with 1 M aq. NaOH solution (100 mL) and the bi-phasic mixture was stirred for 20 min. The layer were then separated, the organic layer was washed with 50 mL of 1 M NaOH, 100 mL of water and was dried with MgSO₄. The mixture was filtered, the filtrate was evaporated in vacuum to dryness. The volatiles were chased out by evaporation of the residue with heptane (2×100 mL). The oily residue was seeded with a few crystals of (R)-tert-butyl(2-chromanyl)methylcarbamate which led to solidifying of the oil. The product was further dried on a oil pump until constant weight. Weight of the isolated material: 34.05 g (99%). Purity 97% (HPLC, 215 nm). Enantiomer ratio R:S. M.p. 62-65° C. (racemate 84-86° C.). The material was used in the next step without further purification.

¹H NMR (300 MHz, CDCl₃), δ: 7.13-7.02 (m, 2H), 6.88-6.77 (m, 2H), 5.03 (br s, 1H), 4.07 (dddd, J=2.1, 3.2, 6.9, 10.9 Hz, 1H), 3.55 (ddd, J=3.2, 6.6, 14.0 Hz, 1H), 3.30 (ddd, J=5.4, 6.9, 14.0 Hz, 1H), 2.87 (ddd, J=6.0, 12.1, 16.4 Hz, 1H), 2.76 (ddd, J=2.4, 5.3, 16.4 Hz, 1H), (dddd, J=2.1, 2.4, 5.3, 6.0 Hz, 1H), (dddd, J=5.3, 10.9, 12.1, 13.5 Hz, 1H), 1.46 (s, 9H).

¹³C NMR (100 MHz, CDCl₃), δ: 156.07, 154.43, 129.57, 127.26, 121.80, 120.39, 116.63, 79.43, 75.20, 44.79, 28.41, 24.78, 24.46.

HRMS (ESI+), m/z: 286.14214 (M+Na),⁺ calc'd for C₁₅H₂₁NO₃Na: 286.14136.

### (R)-tert-butyl(8-(2,6-dichlorophenyl)-2-chromanyl)methylcarbamate

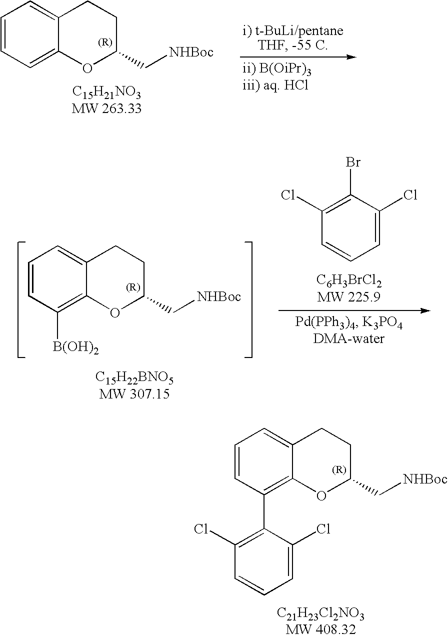

A 1-L flask equipped with a mechanical stirrer, nitrogen inlet, a 500-mL addition funnel capped with a rubber septum, and a thermocouple, set in a acetone bath cooled with a submersible chiller, was purged with nitrogen and charged with a solution of (R)-tert-butyl(2-chromanyl)methylcarbamate (all of the isolated material from the previous step, 0.13 mol) in dry THF (100 mL). THF (500 mL) was added to the flask. The solution was cooled to −60° C. A solution of t-BuLi in pentane (1.6 M, titrated, 245 mL, 0.39 mol, 3.0 eq.) was placed into the addition funnel and from there it was added slowly to the solution in the flask. The temperature was maintained below −57° C. by adjusting the rate of addition, addition time was 50 min.

When the addition was complete, the bath temperature was set at −60° C., the temperature inside the flask equilibrated at −57° C. The mixture was left overnight at that temperature (18 hr). Neat triisopropyl borate (149 mL, 0.65 mol, 5.0 eq.) was placed into the addition funnel. The reaction mixture was chilled to −70° C., and triisopropyl borate was added rapidly to the reaction mixture (exotherm, temperature rose to −52°). The cold bath was removed, and the reaction mixture was allowed to warm to room temperature. HPLC analysis, area %: boronic acid 92%, borinic acid (Ar₂BOH) 6%, unknown impurity 2%.

A 2-L Erlenmeyer flask was equipped with a mechanic stirrer and a thermocouple and set in an acetone bath was charged with 1 M aq. HCl solution (520 mL). This quench solution was chilled to −5° C. (bath temperature −15° C.). The reaction mixture was transferred to the quench solution via a cannula (gauge 12). The transfer rate was adjusted to keep the temperature below 0° C. When the addition was complete, the bath was removed and the resulting biphasic mixture was stirred at room temperature for 2 hours.

The layers were separated. The organic layer was evaporated in vacuo to a thick oil. The aqueous layer was extracted with MTBE (2×100 mL). The extracts were combined with the residue after evaporation and the resulting solution was washed with brine (2×50 mL) and evaporated in vacuum to afford a semi-solid white residue (61.7 g).

The residue was dissolved dimethylacetamide (DMA, 300 mL) and the solution was placed into a 1-L round bottom flask equipped with a mechanical stirrer, a thermocouple and a nitrogen line. Solid 2,6-dichlorobromobenzene (27.9 g, 0.124 mol, 0.95 eq) and potassium phosphate (83 g, 0.39 mol, 3.0 eq.) were added. The reaction flask was purged with nitrogen, then solid Pd(PPh3)4 (3.00 g, 2.6 mmol, 2 mol %) was added. The reaction mixture was heated at 100° C. for 18 hours. The HPLC analysis at that point showed no boronic acid remaining in the mixture.

The reaction mixture was allowed to cool to room temperature, then it was transferred to a mixture of 1.1 L of water and 100 mL of heptane which was mechanically stirred in a 2-L Erlenmeyer flask. The resulting mixture was stirred at room temperature for 3 hours, then the solids were filtered off and washed with heptane and water. The cake was dried on the filter, 41.1 g (76% from the amide, 5 steps) as an off-white solid. M.p. 158-161° C. (racemate 132-136° C.).

¹H NMR (400 MHz, CDCl₃), δ: 7.42-7.37 (m, 2H), 7.22 (t, J=7.1 Hz, 1H) 7.13 (m, 1H), 7.01-6.93 (m, 2H), 4.70 (m, 1H), 4.07 (m, 1H), 3.42 (ddd, J=3.2, 7.2, 13.9 Hz, 1H), 3.12 (ddd, J=5.1, 7.4, 13.9 Hz, 1H), 2.94 (ddd, J=6.3, 11.2, 16.7 Hz, 1H), 2.84 (ddd, J=3.2, 5.9, 16.7 Hz, 1H), 1.98 (d5, J=2.8, 13.6 Hz, 1H), 1.85-1.73 (m, 1H), 1.43 (s, 9H).

¹³C NMR (100 MHz, CDCl₃), δ: 156.05, 151.38, 136.48, 135.59, 135.32, 130.01, 128.84, 128.55, 127.88, 127.68, 125.00, 122.29, 120.00, 79.12, 75.21, 44.38, 28.38, 24.58, 24.37.

HRMS (ESI+), m/z: 430.09447 (M+Na),⁺ calc'd for C₂₁H₂₃Cl₂NO₃Na: 430.09472.

### (R)-2-(Aminomethyl)-8-(2,6-dichlorophenyl)chromane hydrochloride

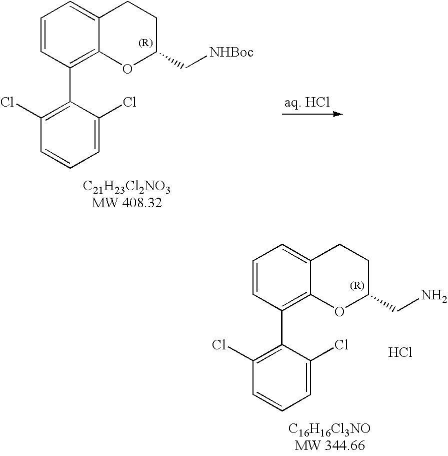

In a 500-mL round-bottom flask, equipped with a magnetic stirrer, a temperature probe, and a reflux condenser, (R)-tert-butyl(8-(2,6-dichlorophenyl)-2-chromanyl)methylcarbamate (47.06 g, 115 mmol) was suspended in 250 ml of methanol, and to the suspension were added 70 ml of conc. aqueous HCl (exotherm to 36° C.). The resulting suspension was heated at reflux for 40 min; most of the material dissolved, HPLC showed complete conversion. The reaction mixture was cooled to 30-35° C. and filtered through a glass filter. The insoluble residue was washed with methanol. The filtrate was evaporated in vacuum; the product crystallized upon concentration. The crude crystalline mass was diluted with water-methanol 3:1 mixture (100 mL), cooled in an ice bath, filtered, washed with cold water, and dried on the Teller machine to yield 29.3 g (74%) of the title compound. Additional 2.5 g crystallized out of the filtrate; the total yield was 80%.

The crude material (32.4 g) was dissolved in 45 mL of absolute ethanol at 56° C.; the solution was cooled down to 40° C. and diluted slowly with 450 mL of diethyl ether. The crystalline product was filtered, washed with ether, and dried in vacuum overnight at room temperature to yield 29.2 g (90%) of the title compound; analytical purity 97.4%, optical purity 99.9%.

The recrystallization was repeated as described using 50 mL of ethanol and 450 mL of ether. Yield 27.4 g (94%) as white crystals. M.p. 211-212° C.

Analytical purity 99.0% (HPLC 215 nm). Impurities 0.1%, 0.7%.

Enantiomeric purity 99.8:0.2 (99.6% ee). HPLC conditions: Chiralpak ASH, 0.46×25 cm column, isocratic 95% Heptane/DEA, 5% ethanol mobile phase, 1.0 mL/min flow rate, distomer 9.4 min, eutomer 10.5 min.

¹H NMR (dmso-d₆, 400 MHz), δ: 8.19 (br s, 3H), 7.55 (d, J=8.5 Hz, 2H), 7.40 (dd, J=8.5, 8.5, 1H), 7.18 (dd, J=7.3, 2.0, 1H), 6.97 (dd, J=7.6, 7.3, 1H), 6.94 (dd, J=7.6, 2.0, 1H), 4.27 (m, 1H), 2.91 (m, 1H), 2.95 (m, 1H), 2.87 (m, 2H), 2.16 (m, 1H), 1.76 (m, 1H).

¹³C NMR (dmso-d₆, 100 MHz), δ: 150.45, 135.60, 134.39, 134.36, 130.02, 129.71, 128.52, 128.07, 127.98, 124.24, 122.26, 120.16, 72.21, 41.63, 23.76, 22.90.

ES+MS, m/z: 308.1 (MH⁺). [α]²⁵_{D}=−1.5°.

Anal. Calcd for C₁₆H₁₆Cl₃NO: C, 55.76; H, 4.68; N, 4.06. Found: C, 55.15; H, 4.89; N, 4.04.

### Example 2

### Screening of Catalysts and Conditions for the Asymmetric Hydrogenation of 4-oxo-4H-1-benzopyran-2-carboxylic acid

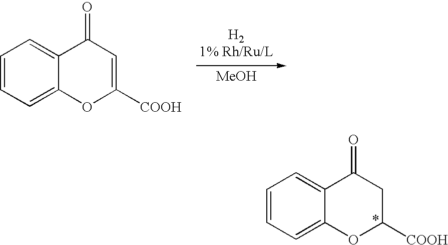

The glass vessels of Argonaut Endeavor™ parallel hydrogenator were independently charged under nitrogen with 4-oxo-4H-1-benzopyran-2-carboxylic acid (0.05 g), methanol (2 mL), and a solution containing the catalyst (2 mL). The catalysts were prepared by dissolving bis(norbornadiene)rhodium(I) tetrafluoroborate (RhNBD₂BF₄) or bis(2-methylallyl)(1,5-cyclooctadiene)ruthenium(II) (RuCODMetAll₂) and a ligand in methanol (2 mL) and aging the solution for 1 hour. The mixtures were subjected to hydrogen pressure of 50-300 psi and temperature of 30-50° C. for 12-36 hours. Ligands: SL-W008-2; SL-J002-1; R-BINAP; SL-A001-2; SL-W008-1. Metal sources: bis(norbornadiene)rhodium(I) tetrafluoroborate; bis(2-methylallyl)(1,5-cyclooctadiene)ruthenium(II).

### Example 3

### Cleavage of the 4-carbonyl Moiety by Hydrogenolysis (Racemic Model)

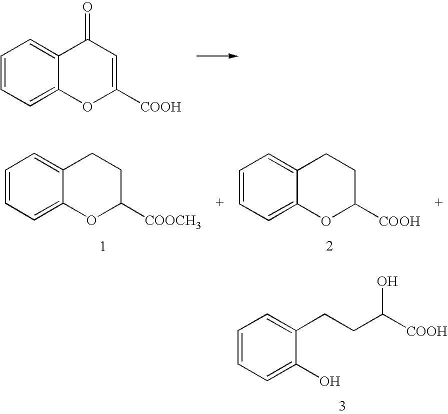

The 4-carbonyl moiety of 4-oxo-4H-1-benzopyran-2-carboxylic acid did not reduce under the homogenous catalysis conditions, only the double bond. The moiety was cleaved in the presence of palladium on carbon under acidic conditions. When methanol was used as the solvent, methyl ester was the main product (e.g. 1). This ester can be elaborated to the amide without intermediary hydrolysis. When acetic acid was used, the chroman-2-carboxylic acid (e.g. 2) was the sole product.

Experiments per formed in a manner similar to that described in Example 1 (50° C., 500 rpm, 12 hours, 2 drops of an acid per each vessel), using 5% Pd/C (Degussa type E1002 XU/W, 50% wet).

### Example 4

### Asymmetric Hydrogenation of 4-oxo-4H-1-benzopyran-2-carboxylic acid to (S)-4-oxochroman-2-carboxylic acid

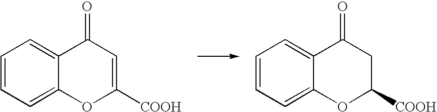

The glass vessels of Argonaut Endeavor™ parallel hydrogenator were independently charged under nitrogen with 4-oxo-4H-1-benzopyran-2-carboxylic acid (0.05 g), methanol (3 mL), and a solution containing the catalyst (1 mL). The catalyst was prepared by dissolving bis(norbornadiene)rhodium(I) tetrafluoroborate (11.9 mg) and Solvias AG, SL-W008-2 ligand (27.7 mg) in methanol (8 mL) and aging the solution for 1 hour. The mixtures were then subjected to hydrogen pressure of 50 psi and temperature 30-50° C. for 36 hours.

Upon completion, analytical samples were withdrawn, solvent evaporated, the residues dissolved in DMSO-d₆, and solutions analyzed by ¹H NMR. The spectra revealed absence of the starting material (singlet at 6.9 ppm, CH═C).

For the enantiomeric excess (% ee) determination using chiral HPLC, 250-μL reaction samples were withdrawn, methanol was evaporated, and the residues were dissolved in isopropanol (1 mL).

The reaction mixtures were combined and solvent evaporated to give (S)-4-oxochroman-2-carboxylic acid as crystalline solid (0.428 g). Optical purity 80.8% ee (LC). HPLC (area %): 90.4 (RT 6.6 min.), 9.6 (RT 8.9 min.). GC/MS: RT 9.40 min., m/z 192 (133, 100%). ¹H NMR (DMSO-d₆): methine proton at C2, 5.31 ppm (dd, J=7.35, J=5.31 Hz, 1H).

HPLC conditions and determination of the absolute configuration of 4-oxochroman-2-carboxylic acid.
Column: Chiralcel OD-H 150×4.6 mm, 5 micron
Mobil phase: 900 mL Hexane, 100 mL IPA, 2 mL TFA
Flow Rate: 1.0 mL/min.
Detection Wavelength: 247 nm
Column Temperature: 35° C.
Sample Diluent: EtOH
Sample Concentration: 3 mg/mL
Injection Volume: 5 μL
Run Time: 20 min.

Retention Time: S-enantiomer 6.6 min.; R-enantiomer 8.9 min.

### Example 5

### Preparation of (2S-3,4-dihydro-2H-1-benzopyran-2-carboxylic acid

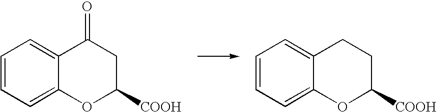

A solution of (S)-4-oxochroman-2-carboxylic acid (0.259 g) in glacial acetic acid (8 mL), two drops of concentrated sulfuric acid, and 5% palladium on carbon catalyst (21 mg, Degussa type E1002 XU/W, 50% wet) were subjected to hydrogen pressure (60 psi) in the Argonaut Endeavor™ parallel hydrogenator (500 rpm, 50° C., 12 h). The reaction mixture was filtered through an Acrodisc™ filter and the solvent evaporated to give (2S)-3,4-dihydro-2H-1-benzopyran-2-carboxylic acid a solid residue (0.191 g). Optical purity 85.7% ee (LC). HPLC (area %): 92.9 (RT 2.6 min.), 7.1 (RT 6.3 min.). GC/MS: RT 10.19 min., m/z 178 (133, 100%). ¹H NMR (DMSO-d₆): methine proton at C2, 4.76 (dd, J=6.43, J=3.96 Hz, 1H).

HPLC conditions and determination of the absolute configuration of 3,4-dihydro-2H-1-benzopyran-2-carboxylic acid were performed as follows:
Column: Chiralcel OD-H 150×4.6 mm, 5 micron
Mobil phase: 850 mL Hexane, 100 mL IPA, 50 mL MeOH, 2 mL TFA
Flow Rate: 1.5 mL/min.
Detection Wavelength: 220 nm
Column Temperature: 35° C.
Sample Diluent: EtOH
Sample Concentration: 3 mg/mL
Injection Volume: 5 μL
Run Time: 10 min.
Retention Time: S-enantiomer 2.6 min.; R-enantiomer 6.2 min.

### Example 6

### Asymmetric Hydrogenation of 4-oxo-4H-1-benzopyran-2-carboxylic acid to (R)-4-oxochroman-2-carboxylic acid

In an analogous manner as described in Example 4 the (R) enantiomer of 4-oxochroman-2-carboxylic acid was prepared in 77.6% ee by substituting SL-W008-2 ligand with its enantiomer, SL-W008-1. LC: RT 8.8 min.

The Two Solvias Ligands

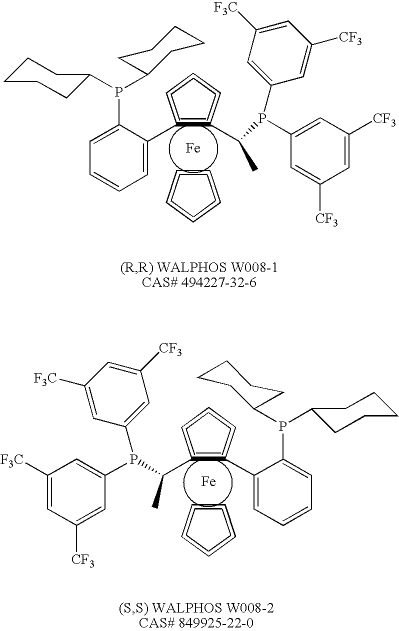

### Example 7

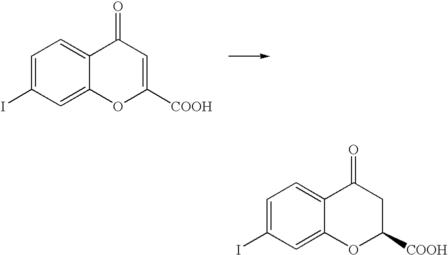

### Asymmetric Hydrogenation of 7-iodo-4-oxo-4H-1-benzopyran-2-carboxylic acid to (S)-7-iodo-4-oxochroman-2-carboxylic acid

In a similar manner as described in Example 4, 7-iodo-4-oxo-4H-1-benzopyran-2-carboxylic acid (0.075 g each vessel) was hydrogenated (50-150 psi pressure) in the presence of asymmetric catalysts at 50° C. for 18 hours. HPLC RT: S-enantiomer 7.2 min.; R-enantiomer 7.7 min. ¹H NMR (DMSO-d₆): methine proton at C2, 5.46 (t, J=6.03 Hz, 1H).

### Example 8

### An Alternate Synthetic Route in the Preparation of (R)-2-Aminomethyl)-8-(2,6-dichlorophenyl)chromane hydrochloride

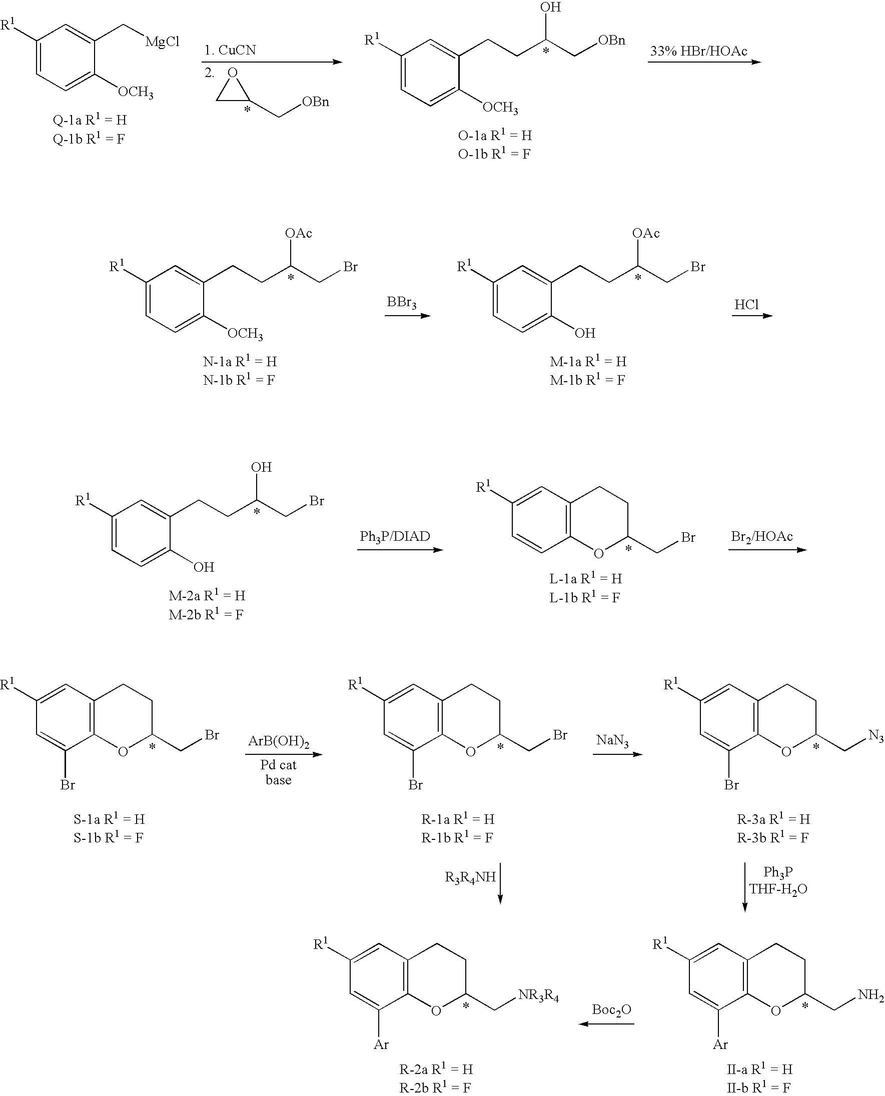

### Example 9

### Preparation of (S)-1Benzyloxy)-4-(2-methoxyphenyl)butan-2-ol (O-1a)

To a solution of 2-methoxybenzylmaganisum chloride (Rieke, 100 mL, 0.25 M) in anhydrous tetrahydrofuran was added a suspension of copper cyanide (1.1 g) in anhydrous tetrahydrofuran at −30° C. The resulting mixture was stirred at −30 to −25° C. for 1 hour. Optical active glycidyl benzoether (1.9 mL, 12.5 mmol) was introduced at −25° C. The reaction mixture was stirred at −25° C. to 0° C. for 4 hours, then the bath was removed. The reaction mixture was stirred at room temperature for 3 days. The reaction mixture was poured into saturated ammonium chloride-water and extracted with methylene chloride. The solvent was removed under vacuum. ISCO CombiFlash® chromatography with 0-30% ethyl acetate in hexane afforded desired product 4.4 g as a colorless oil. MS ES m/e 309.1; ¹HNMR (400 MHz, DMSO-d₆) indicated the desired product contained trace of (2-methoxyphenyl)methanol.

### Example 10

### Preparation of (S)-1-Benzyloxy)-4-(5-fluoro-2-methoxyphenyl)butan-2-ol (O-1b)

Starting with 5-fluoro-2-methoxybenzylmaganisum chloride (Rieke, 100 mL, 0.25 M) and following the procedure described in Example 10 gave the desired product as a colorless oil. ¹HNMR (400 MHz, DMSO-d₆) indicated the desired product contained trace of (5-fluoro-2-methoxyphenyl)methanol.

### Example 11

### Preparation of (S)-1-Bromo-4-(2-methoxy-phenyl)butan-2-yl acetate (N-1a)

(S)-1-Benzyloxy-4-(2-methoxyphenyl)butan-2-ol (1.0 g) was dissolved in 33% hydrogen bromide in acetic acid (12 mL). The reaction mixture was heated at 55° C. for 40 minute. The solvent was removed under vacuum. The residue was dissolved in methylene chloride and washed with ammonium hydroxide-water. The organic solvent was removed under vacuum. ISCO CombiFlash® chromatography with 0-30% ethyl acetate in hexane afforded the desired product 0.67 g (64%) as a light brown oil. MS EI m/e 300 [M]⁺, [α]=−7° (1% solution in MeOH).

### Example 12

### Preparation of (S)-1-Bromo-4-(5-fluoro-2-methoxy-phenyl)butan-2-yl acetate (N-1b)

Starting with (S)-1-(benzyloxy-4-(5-fluoro-2-methoxyphenyl)butan-2-ol and following the procedure described in Example 11 gave the desired product 1.3 g as a colorless oil. ¹HNMR (400 MHz, DMSO-d₆) δ 1.80-1.94 (m, 2H), 1.99 (s, 3H), 2.50-2.55 (m, 2H), 3.62-3.66 (m, 2H), 3.71 (s, 3H), 4.83-4.86 (m, 1H), 6.87-6.97 (m, 3H).

### Example 13

### Preparation of (S)-1-Bromo-4-(2-hydroxy-phenyl)butan-2-yl acetate (M-1a)

To a solution of (S)-1-bromo-4-(2-methoxy-phenyl)butan-2-yl acetate (0.7 g, 2.3 mmol) in methylene chloride was added boron tribromide (0.33 mL, 3.5 mmol) at −78° C. The resulting mixture was stirred at −78° C. to room temperature overnight. The reaction mixture was poured in the ice-NH₄OH and extracted with methylene chloride. The organic layer was washed with water and dried over anhydrous sodium sulfate and filtered. The solvent was removed under vacuum. ISCO CombiFlash® chromatography with 10-40% ethyl acetate in hexanes afforded 0.42 g (100%) of the title product as a colorless oil. MS EI m/e 286 [M]⁺, [α]=−2° (1% solution in MeOH).

### Example 14

### Preparation of (S)-1-Bromo-4-(5-fluoro-2-hydroxy-phenyl)butan-2-yl acetate (M-1b)

Starting with (S)-1-bromo-4-(5-fluoro-2-methoxy-phenyl)butan-2-yl acetate (1.3 g, 4.1 mmol) and following the procedure described in Example 14 gave the desired product 0.80 g (65%) as a colorless oil. ¹HNMR (400 MHz, DMSO-d₆) δ 1.80-1.86 (m, 2H), 1.99 (s, 3H), 2.47-2.52 (m, 2H), 3.58-3.61 (m, 2H), 4.85-4.87 (m, 1H), 6.68-6.72 (m, 1H), 6.75-6.80 (m, 1H), 6.83-6.86 (m, 1H), 9.27 (s, 1H).

### Example 15

### Preparation of (S)-2-(Bromo-3-hydroxybutyl)phenol (M-2a)

To a solution of (S)-1-bromo-4-(2-hydroxy-phenyl)butan-2-yl acetate (2.03 g, 7.1 mmol) in methanol was added hydrogen chloride in ether (1.0 M, 21.2 mL, 21.3 mmol) at room temperature. The mixture was stirred at room temperature overnight. The solvent was removed under vacuum. ISCO CombiFlash® chromatography with 0-30% ethyl acetate afforded 1.31 g (76%) of the title product as a colorless oil. MS APPI m/z 243 [M−H]⁻, [α]=−27° (1% solution in MeOH).

### Example 16

### Preparation of (S-2-Bromo-3-hydroxybutyl)-4-fluorophenol (M-2b)

Starting with (S)-1-bromo-4-(5-fluoro-2-hydroxy-phenyl)butan-2-yl acetate (0.80 g, 1.6 mmol) and following the procedure described in Example 15 gave the desired product 0.43 g (63%) as a colorless oil. MS EI m/z 262 M⁺.

### Example 17

### Preparation of (R)-2-(Bromomethyl)chroman (L-1a)

To a solution of (S)-2-(bromo-3-hydroxybutyl)phenol (1.31 g, 5.3 mmol) in tetrahydrofuran was added triphenyl phosphine (5.2 g, 20 mmol) and followed by DIAD (2.6 mL, 13.2 mmol) at room temperature. The reaction mixture was stirred at room temperature for 45 minute. Solvent was removed under vacuum. ISCO CombiFlash® chromatography with 0-5% ethyl acetate afforded 1.05 g (82%) of the title product as a colorless oil. MS EI m/z 226 [M]⁺, [α]=−95.0° (c 1% solution in MeOH)

### Example 18

### Preparation of (R)-2-(Bromomethyl)-6-fluorochroman (L-1b)

Starting with (S-2-bromo-3-hydroxybutyl)-4-fluorophenol (0.43 g, 1.6 mmol) and following the procedure described in Example 17 gave the desired product 0.30 g (75%) as a colorless oil. MS APPI m/z 244 [M]⁺, [α]=−92° (1% solution in MeOH).

### Example 19

### Preparation of (R)-8-Bromo-2-bromomethyl)-6-fluorochroman (S-1b)

To a solution of (R)-(2-bromomethyl)-6-fluoro-chroman (0.3 g, 1.2 mmol) in acetic acid was added bromine (0.13 mL, 2.4 mmol) at room temperature. The mixture was stirred at room temperature overnight. The solvent was removed under the vacuum and the residue was washed with Na₂SO₃ and extracted with methylene chloride. ISCO CombiFlash® chromatography with 0-10% ethyl acetate in hexanes afforded 0.32 g (81%) of the title product as a light yellow oil. MS APPI m/z 322 [M]⁺, [α]=−120° (1% solution in MeOH).

### Example 20

### Preparation of (2R)-2-(Bromomethyl)-6-fluoro-8-(2-trifluoromethyl)phenyl-chroman (R-1b)

To a solution of (R)-8-bromo-2-bromomethyl)-6-fluorochroman (0.16 g, 0.49 mmol) and 2-trifluorobenzene boronic acid (0.4 g, 2 mmol) in dioxane-water (4/1) was added dichlorobis(tri-o-tolyphosphine)-palladium (0.2 g, 0.02 mmol) and potassium carbonate (0.17 g, 1.2 mmol) at 90° C. The mixture was heated at 90° C. for 3 hours. The mixture was filtered through a pad of celite and concentrated under vacuum. ISCO CombiFlash® chromatography with 0-40% ethyl acetate in hexanes afforded 0.11 g (57%) of the title product as a colorless oil. MS APPI m/z 388 M⁺.

### Example 21

### Preparation of (2R)-2-(Bromomethylfluoro-6-fluoro-8-(4-methoxy-2-methyl)phenyl-chroman. (R-1b)

Starting with (R)-8-bromo-2-bromomethyl)-6-fluorochroman (0.16 g, 0.49 mmol) and 4-methoxy-2-methyl benzene boronic acid (0.27 g, 2 mmol) following the procedure described in Example 20 gave arise the desired product 0.09 g (50%) as a colorless oil. ¹HNMR (400 MHz, DMSO-d₆) δ 1.67-1.78 (m, 1H), 1.97-2.01 (m, 1H), 2.07 (s, 3H), 2.82-2.93 (m, 1H), 2.77-2.80 (m, 1H), 3.53 (m, 1H), 3.71 (s, 3H), 4.13-4.15 (m, 1H), 6.66-6.72 (m, 2H), 6.76 (m, 1H), 6.89 (m, 1H), 6.90 (m, 1H).

### Example 22

### Preparation of (2R)-2-(Azidomethyl)-6-fluoro-8-(2-trifluoromethyl)phenylchroman (R-3b)

To a solution of (2R)-2-(bromomethyl)-6-fluoro-8-2-trifluoromethyl)phenyl-chroman (0.11 g, 0.28 mmol) in DMF was added sodium azide (0.11 g, 0.28 mmol). The mixture was heated at 90° C. overnight. The reaction was quenched with water. The mixture was extracted with methylene chloride. The organic layer was washed with water and dried over sodium sulfate. The organic solvent was removed under vacuum. ISCO CombiFlash® chromatography with 10% ethyl acetate in hexanes afforded 70 mg (71%) of the title product as a colorless oil. MS EI M⁺ 351.

### Example 23

### Preparation of (2R)-2-(Azidomethyl)-6-fluoro-8-(4-methoxy-2-methyl)phenylchroman (R-3b)

Starting with (2R)-2-(bromomethyl)-6-fluoro-8-(4-methoxy-2-methyl)phenyl-chroman (0.09 g, 0.25 mmol) following the procedure described in Example 22 gave rise the desired product 0.08 g (100%) as a colorless oil. MS ES m/z 328.1 [M+H]⁺; [α]=−4° (c 1% solution in MeOH).

### Example 24

### Preparation of 1-{(2R)-6-fluoro-8-[2-trifluoromethyl)phenyl]-3,4-dihydro-2H-chromen-2-yl}methanamine (II-b)

To a solution of (2R)-2-(azidomethyl)-6-fluoro-8-(2-trifluoromethyl)phenylchroman (70 mg, 0.2 mmol) in tetrahydrofuran was added polymer-supported triphenylphosphine (”3 mmol/g, 0.4 mmol) and water. The mixture was stirred at room temperature for 1-2 days, and filtered through a pad of celite. The solvent was removed under vacuum to form a colorless oil. The oil was dissolved in ethyl acetate and made into its hydrochloride salt as an off-white solid. mp 174-176° C. [α]_{D}²⁵ =−4.00° (c=1% SOLUTION, DMSO); MS (APPI) m/z 326;HRMS: calcd for C₁₇H₁₅F₄NO+H⁺, 326.11625; found (ESI, [M+H]⁺), 326.1172.

### Example 25

### Preparation of 1-[(2R)-6-Fluoro-8-(4-methoxy-2-methylphenyl)-3,4-dihydro-2H-chromen-2-yl]methanamine (II-b)

Staring with (2R)-2-(azidomethyl)-6-fluoro-8-(4-methoxy-2-methyl)phenylchroman (0.08 g, 0.24 mmol) following the procedure described for Example 24 gave arise the desired product as a colorless oil. The oil was dissolved in ethyl acetate and made into its hydrochloride salt as a white solid. mp 159-161° C.; [α]_{D}²⁵=−68.0° (c=1% SOLUTION, MeOH); MS (ES) m/z 302.1; HRMS: calcd for C₁₈H₂₀FNO₂+H⁺, 302.15508; found (ESI, [M+H]⁺), 302.1552.

### Example 26

### Preparation of (R)-2-(Azidomethyl)chroman (R-3a)

To a solution of (R)-2-(bromomethyl)chroman (1.05 g, 4.6 mmol) in DMF was added sodium azide (1.5 g, 23 mmol). The mixture was heated at 90° C. overnight. The reaction was quenched with water. The mixture was extracted with methylene chloride. The organic layer was washed with water and dried over sodium sulfate. The organic solvent was removed under vacuum. ISCO CombiFlash® chromatography with 10% ethyl acetate in hexanes afforded 0.87 g (99%) of the title product as a colorless oil. MS EI m/z 189 M⁺, [α]=−72° (c 1% solution in MeOH).

### Example 27

### Preparation of (R)-Chroman-2-ylmethanamine (II-a)

To a solution of (R)-2-(azidomethyl)chroman (1.0 mmol) in tetrahydrofuran was added polymer-supported triphenylphosphine (˜3 mmol/g, 2.0 mmol) and water. The mixture was stirred at room temperature for 1-2 days, and filtered through a pad of celite. The solvent was removed under vacuum to form a cololess oil. The oil was dissolved in ethyl acetate and made into its hydrochloride salt as an off-white solid. MS APPI m/z 164 [M+H]⁺.

### Example 28

### Preparation of (R)-tert-Butyl chroman-2-ylmethylcarbamate (R-2a)

To a solution of (R)-chroman-2-ylmethanamine (0.56 g, 3.4 mmol) in methylene chloride (20 mL) was add di-tert-butyldicarbonate at 0° C. The mixture was stirred at 0° C. to room temperature in overnight period. The solvent was removed under vacuum. ISCO CombiFlash® chromatography with 5-10% ethyl acetate in hexanes afforded 1.12 g (80%) of the title product as a colorless oil. MS EI 263 M⁺, [α]=−111° (c 6.1 mg/0.7 ml, MeOH).

We claim:

1. A method for preparing a compound of formula II: 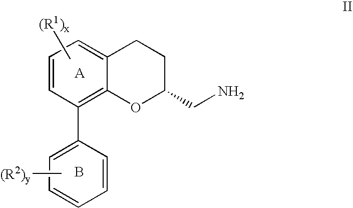
wherein:
x is 0-3;
y is 0-5;
each R¹ is independently —R, —CN, halogen or —OR;
each R is independently hydrogen, C₁₋₆ aliphatic or C₁₋₆ haloaliphatic; and
each R² is independently -Ph, halogen, —CN, —R or —OR,
comprising the steps of:
(a) providing a compound of formula A: 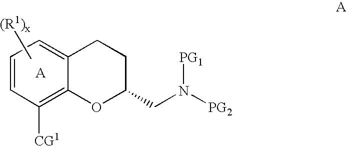
wherein:
x is 0-3;
each R¹ is independently —R, —CN, halogen or —OR;
each R is independently hydrogen, C₁₋₆ aliphatic or C₁₋₆ haloaliphatic;
PG¹ and PG² are each independently hydrogen or suitable amino protecting groups; and
CG¹ is a coupling group that facilitates transition metal-mediated Cₛₚ₂-Cₛₚ₂ coupling between the attached Cₛₚ₂ carbon and a Cₛₚ₂ carbon bearing a CG² coupling group,
(b) coupling said compound of formula A with a compound of formula B: 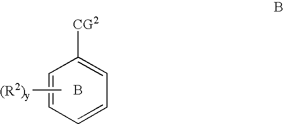
wherein:
y is 0-5;
each R is independently hydrogen, C₁₋₆ aliphatic or C₁₋₆ haloaliphatic;
each R² is independently -Ph, halogen, —CN, —R or —OR; and
CG² is a coupling group that facilitates transition metal-mediated Cₛₚ₂-Cₛₚ₂ coupling between the attached Cₛₚ₂ carbon and a Cₛₚ₂ carbon bearing a CG¹ coupling group;
in the presence of a suitable transition metal,
and
(c) deprotecting the optionally protected amine moiety of the coupling product to form a compound of formula II.

2. The method according to claim 1, wherein CG¹ is a boronic acid, a boronic ester, or a borane.

3. The method according to claim 2, wherein CG² is Br, I, or OTf.

4. The method according to claim 3, wherein the compound of formula A is 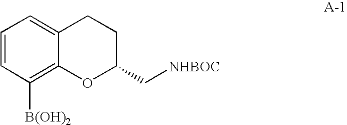
and the compound of formula B is

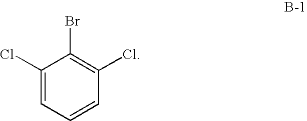

5. The method according to claim 1, further comprising the steps of:
(a) providing a compound of formula C: 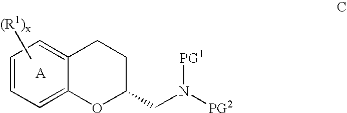
wherein:
x is 0-3;
each R¹ is independently —R, -Ph, —CN, halogen, or —OR;
each R is independently hydrogen, C₁₋₃ aliphatic or C₁₋₃ fluoroaliphatic; and
PG¹ and PG² are each independently hydrogen or a suitable amino protecting group,
and
(b) introducing a CG¹ group at the open ortho position relative to the sp2-hybridized carbon bearing the chromane oxygen in formula C to afford a compound of formula A.

6. The method according to claim 5, further comprising the steps of:
(a) providing a compound of formula D: 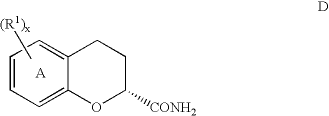
wherein:
x is 0-3;
each R¹ is independently —R, —CN, halogen or —OR; and
each R is independently hydrogen, C₁₋₆ aliphatic or C₁₋₆ haloaliphatic;
(b) reducing the amide moiety in the compound of formula D to the amine;
and
(c) protecting the amine moiety resulting from the reduction of the amide moiety in the compound of formula D with a suitable amine protecting group to afford a compound of formula C.

7. The method according to claim 6, wherein the reduction step (b) is performed with Red-Al[sodium bis(2-methoxyethoxy)aluminumhydride] or lithium aluminum hydride.

8. The method according to claim 6, further comprising the steps of:
(a) providing a compound of formula E: 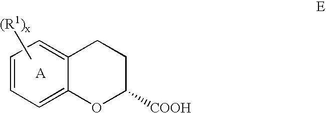
wherein:
x is 0-3;
each R¹ is independently —R, —CN, halogen or —OR; and
each R is independently hydrogen, C₁₋₆ aliphatic or C₁₋₆ haloaliphatic;
and
(b) converting the carboxylic acid moiety in the compound of formula E into an amide moiety to form a compound of formula D.

9. The method according to claim 8, wherein step (b) is conducted by first activating the carboxylic acid to facilitate acylation and subsequently treating the activated species with a source of ammonia.

10. The method according to claim 8, further comprising the steps of:
(a) providing a compound of formula F: 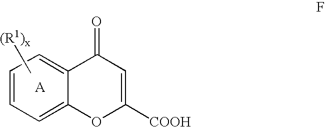
wherein:
x is 0-3;
each R¹ is independently —R, —CN, halogen or —OR; and
each R is independently hydrogen, C₁₋₆ aliphatic or C₁₋₆ haloaliphatic;
and
(b) hydrogenating the compound of formula F to afford a compound of formula E.

11. The method according to claim 10, wherein step (b) comprises subjecting the compound of formula F to non-asymmetric hydrogenation conditions, forming diastereomeric salts by adding an enantioenriched chiral amine to the racemic hydrogenation product E-1, selectively crystallizing one of the diastereomeric salts to afford a diastereomerically enriched mixture of salts, and recovering the compound of formula E in enantioenriched form.

12. The method according to claim 11, wherein the compound of formula F is 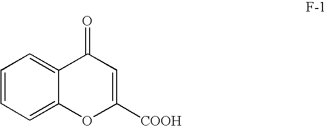
and the enantioenriched chiral amine is (R)-1-phenylpropylamine.

13. The method according to claim 10, wherein hydrogenation in step (b) is performed in an asymmetric fashion.

14. The method according to claim 12, wherein step (b) is catalyzed by a suitable chiral catalyst.

15. The method according to claim 10, further comprising the steps of:
(a) providing a compound of formula G: 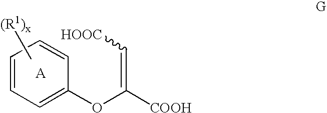
wherein:
x is 0-3;
each R¹ is independently —R, —CN, halogen or —OR; and
each R is independently hydrogen, C₁₋₆ aliphatic or C₁₋₆ haloaliphatic;
and
(b) cyclizing the compound of formula G to afford a compound of formula F.

16. The method according to claim 15, further comprising the steps of:
(a) providing a compound of formula H: 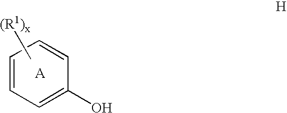
wherein:
x is 0-3;
each R¹ is independently —R, —CN, halogen or —OR; and
each R is independently hydrogen, C₁₋₆ aliphatic or C₁₋₆ haloaliphatic;
(b) allowing said compound of formula H to react with a compound of formula J: 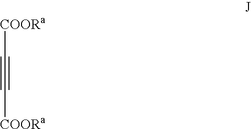
wherein:
x is 0-3;
each R¹ is independently —R, —CN, halogen or —OR;
each R is independently hydrogen, C₁₋₆ aliphatic or C₁₋₆ haloaliphatic; and
each R^{a} is hydrogen, C₁₋₆ aliphatic, phenyl, benzyl, or tri(C₁₋₆ aliphatic)silyl,
and
(c) removing the R^{a} groups from the product of the reaction between the compound of formula H and the compound of formula J to afford a compound of formula G.

17. A method for preparing a compound of formula C: 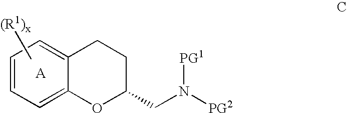
wherein:
x is 0-3;
each R¹ is independently —R, —CN, halogen or —OR;
each R is independently hydrogen, C₁₋₆ aliphatic or C₁₋₆ haloaliphatic; and
PG¹ and PG² are each hydrogen or a suitable protecting group,
comprising the steps of:
(a) providing a compound of formula M: 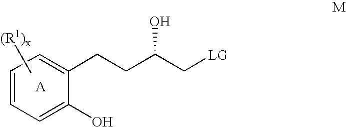
wherein:
x is 0-3;
each R¹ is independently —R, —CN, halogen or —OR;
each R is independently hydrogen, C₁₋₆ aliphatic or C₁₋₆ haloaliphatic; and
LG is a suitable leaving group,
(b) cycling the compound of formula M to form a compound of formula L. 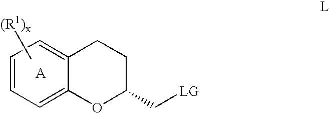
wherein:
x is 0-3;
each R¹ is independently —R, —CN, halogen or —OR;
each R is independently hydrogen, C₁₋₆ aliphatic or C₁₋₆ haloaliphatic; and
LG is a suitable leaving group,
and
(c) treating the compound of formula L with a suitable amine to afford the compound of formula C.

18. The method according to claim 17, further comprising the steps of:
(a) providing a compound of formula N: 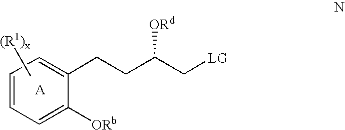
wherein:
x is 0-3;
each R¹ is independently —R, —CN, halogen or —OR;
each R is independently hydrogen, C₁₋₆ aliphatic or C₁₋₆ haloaliphatic;
R^{b} is hydrogen or a suitable hydroxyl protecting group;
R^{d} is hydrogen or a suitable hydroxyl protecting group; and
LG is a suitable leaving group,
and
(b) removing protecting group R^{b} and, if present, R^{d}, to form the diol of formula M 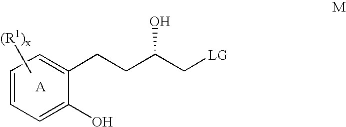
wherein:
x is 0-3;
each R¹ is independently —R, —CN, halogen or —OR;
each R is independently hydrogen, C₁₋₆ aliphatic or C₁₋₆ haloaliphatic; and
LG is a suitable leaving group.

19. The method according to claim 18, further comprising the steps of:
(a) providing a compound of formula O: 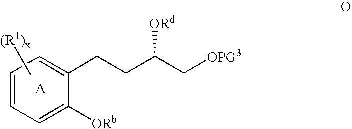
wherein:
x is 0-3;
y is 0-5;
each R¹ is independently —R, —CN, halogen or —OR;
each R is independently hydrogen, C₁₋₆ aliphatic or C₁₋₆ haloaliphatic;
each R² is independently -Ph, halogen, —CN, —R or —OR;
R^{b} is hydrogen or a suitable hydroxyl protecting group;
R^{d} is hydrogen or a suitable hydroxyl protecting group; and
PG³ is a hydroxyl protecting group,
and
(b) removing PG³ and converting the free hydroxyl moiety into a suitable leaving group to afford the compound of formula N.

20. The method according to claim 19, further comprising the steps of:
(a) providing a compound of formula Q: 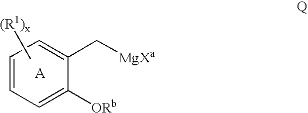
wherein:
x is 0-3;
X^{a} is halogen;
each R¹ is independently —R, —CN, halogen or —OR;
each R is independently hydrogen, C₁₋₆ aliphatic or C₁₋₆ haloaliphatic; and
R^{b} is a suitable hydroxyl protecting group,
and
(b) reacting the compound of formula Q with a non-racemic compound of formula P: 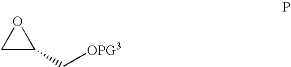
wherein PG³ is a hydroxyl protecting group, to form the compound of formula O.
